(19) United States
(12) Patent Application Publication
(10) Pub. No.: US 2005/0203155 A1
(43) Pub. Date: Sep. 15, 2005

(54) Imidazole compounds and human cellular proteins casein kinase I alpha, delta and epsilon as targets for medical intervention against Hepatitis C Virus infections

(76) Inventors: Konstadinos Salassidis, Eching (DE); Alexander Kurtenbach, Graefelfing (DE); Henrik Daub, Munich (DE); Sabine Obert, Munich (DE); Zoltan Greff, Budapest (HU); Gyorgy Keri, Budapest (HU); Laszlo Orfi, Budapest (HU); Frigyes Waczek, Budapest (HU)

Correspondence Address: J C PATENTS, INC., 4 VENTURE, SUITE 250, IRVINE, CA, 92618, US

(21) Appl. No.: 11/030,538

(22) Filed: Jan. 5, 2005

(30) Foreign Application Priority Data
Jul. 7, 2003 (WO) PCT/EP03/07286
Jul. 5, 2002 (EP) EP 02 015 096.7

(51) Int. Cl. A61K 31/424; A61K 31/4184; A61K 31/4178; C07D 413/02; C07D 43/02

(52) U.S. Cl. 514/375; 514/394; 514/397; 544/216; 548/311.1; 548/307.4; 514/398; 514/367

(57) ABSTRACT

Novel imidazole compounds particularly useful against Hepatitis C Virus infections and diseases associated therewith are described. Applications of the human cellular proteins casein kinase I alpha (α), delta (δ), and epsilon (ε) as targets for medical intervention against Hepatitis C Virus (HCV) infections and diseases are also described. This invention further refers to a method for identifying compounds useful for the prophylaxis and/or treatment of Hepatitis C Virus infections and diseases, methods for treating Hepatitis C Virus infections and diseases, as well as pharmaceutical compositions useful for the prophylaxis and/or treatment of Hepatitis C Virus infections and diseases. Moreover, disclosed are antibodies, oligonucleotides and specific compounds which are effective for the detection, prophylaxis and/or treatment of Hepatitis C Virus infections and diseases. Solid supports useful for the identification of compounds suitable for preventing and/or treating Hepatitis C Virus infections and diseases are also disclosed.

The present invention relates to novel imidazole compounds. The present invention furthermore relates to the human cellular proteins casein kinase I alpha (α), delta (δ), and epsilon (ε) as targets for medical intervention against Hepatitis C Virus (HCV) infections and diseases. Furthermore, the present invention refers to a method for the identification of compounds which are useful for the prophylaxis and/or treatment of infections and diseases caused by Hepatitis C Virus, methods for treating Hepatitis C Virus infections and diseases, as well as pharmaceutical compositions useful for the prophylaxis and/or treatment of Hepatitis C Virus infections and diseases. Moreover, disclosed are antibodies, oligonucleotides and specific compounds which are effective for the detection, prophylaxis and/or treatment of infections and diseases caused by Hepatitis C Virus. In addition, the present invention describes solid supports useful for the identification of compounds suitable for preventing and/or treating infections and diseases caused by said Hepatitis C Virus.

Hepatitis C Virus (HCV) infection is a major cause of chronic hepatitis, cirrhosis and hepatocellular carcinoma. The WHO estimates that approximately 3% of the world population, or 170 million people, have been infected with the Hepatitis C Virus. In the U.S., an estimated 3.9 million Americans have been infected (CDC fact sheet Sep. 00). Over 80% of HCV-infected individuals develop chronic hepatitis, which is associated with disease states ranging from asymptomatic carrier states to repeated inflammation of the liver and serious chronic liver disease. Over the course of 20 years, more than 20% of the chronic HCV-patients are expected to be at risk to develop cirrhosis or progress to hepatocellular carcinoma. Liver failure from chronic hepatitis C is the leading indicator for liver transplantation. Excluding transplantation, the CDC estimates that medical and work-loss cost for HCV annually are around US-$ 600 million. HCV is transmitted primarily by blood and blood products. Due to routine screening of the blood supplies from mid-1992, new transfusion-related cases are exceedingly rare and have been surpassed by injection drug use as the highest risk factor for acquiring the virus. There is also a sexual, however inefficient, route of transmission, and a 6% rate of transmission from infected mothers to their children, which is higher in case of HIV co-infection. In a certain percentage of infections, the mode of transmission remains unknown. In spite of the significant decline in incidence in the 1990's, the number of deaths (estimated deaths annually at the moment: 8000 to 10,000 in U.S.) and severe disease due to HCV is anticipated to triple in the next 10 to 20 years. With respect to the above-mentioned statements see e.g. CDC fact sheet (accessed Dec. 12, 2000); Houghton M. Hepatitis C Viruses, in B N Fields, D M Knipe, P M Howley (ed.) Fields Virology, 1996, Lippencott-Raven Pub., Philadelphia; Rosen H R and Gretch D R, Molecular Medicine Today Vol. 5, 393, September 1999; Science 285, 26, July 1999: News Focus: The scientific challenge of Hepatitis C; Wong J B et al., Am J Public Health, 90, 1562, October 2000: Estimating future hepatitis C morbidity, mortality, and costs in the United States.

According to the Consensus Statement from the EASL (European Association for the Study of the Liver) International Consensus Conference on Hepatitis C (Feb. 26-28, 1999, Paris, France), combination therapy of alpha interferon and ribavirin is the recommended treatment for naive patients. Monotherapy with interferon has also been approved by the U.S. Food and Drug Administration (FDA), but the sustained response rate (HCV RNA remains undetectable in the serum for more than 6 months after end of therapy) is only 15 to 20%, in contrast to 35 to 45% with combination therapy. Interferons (Intron A, Schering-Plough; Roferon A, Hoffmann-LaRoche; Wellferon, Glaxo Wellcome; Infergen, Amgen) are injected subcutaneously three times a week, ribavirin (Rebetol, Schering-Plough) is an oral drug given twice a day. Recommended treatment duration is 6 to 12 months, depending on HCV genotype. Experimental forms of slow-release pegylated interferons (Pegasys, Hoffmann-LaRoche; PEG-Intron, Schering-Plough) have shown improvements in response rates (42 to 82% in combination with ribavirin) and application (once-weekly injection) in recent clinical studies (Hepatology 32:4, Pt 2 of 2. October 2000; NEJM 343, 1673, December 2000; NEJM 343, 1666, December 2000). Common side effects of interferon therapy include: fatigue, muscle aches, head aches, nausea, fever, weight loss, irritability, depression, bone marrow suppression, reversible hair loss. The most common side effects of ribavirin are anemia, fatigue and irritability, itching, skin rash, nasal stuffiness, sinusitis, cough. More serious side effects of mono-and combination therapy occur in less than two percent of patients (NIDDK information: Chronic Hepatitis C: Current Disease Management, accessed 09. December 1999). Some of the contraindications to interferon are psychosis or severe depression; neutropenia and/or thrombocytopenia; organ transplantation except liver; symptomatic heart disease; decompensated cirrhosis; uncontrolled seizures. Contraindications to ribavirin are end-stage renal failure; anemia; hemoglobinopathies; severe heart disease; pregnancy; no reliable method of contraception (consensus statement EASL).

Experimental treatments that are not new forms of interferon are Maxamine (histamine dihydrochloride, Maxim Pharmaceuticals), which will be combined with Interferon in phase III studies, VX-497 (Vertex Pharmaceuticals), an IMP dehydrogenase inhibitor, as a less toxic ribavirin substitute in phase II, and amantadine (Endo Labs), an approved influenza drug, as the third component in triple therapy (phase II). Inhibitors for HCV enzymes such as protease inhibitors, RNA polymerase inhibitors, helicase inhibitors as well as ribozymes and antisense RNAs are under preclinical development (Boehringer Ingelheim, Ribozyme Pharmaceuticals, Vertex Pharmaceuticals, Schering-Plough, Hoffmann-LaRoche, Immusol, Merck etc.). No vaccine is available for prevention or therapeutic use, but several companies are trying to develop conventional or DNA vaccines or immunostimulatory agents (e.g. Chiron, Merck/Vical, Epimmune, NABI, Innogenetics).

In summary, the available treatment for chronic Hepatitis C is expensive, effective only in a certain percentage of patients and adverse side effects are not uncommon.

It is therefore the object of the present invention to provide novel compounds for the prophylaxis and/or treatment of Hepatitis C Virus infections and diseases caused by such infections. Furthermore, it is the object of the present invention to provide targets for detection, medical intervention, prophylaxis and/or treatment of Hepatitis C Virus infections, including acute and chronic Hepatitis C, as well as methods for identifying compounds which can be used for the prophylaxis and/or treatment of Hepatitis C Virus infections. Moreover, it is the object of the present invention to provide agents and compounds detected by these methods for the prophylaxis and/or treatment of Hepatitis C Virus infections, including acute and chronic Hepatitis C, and methods for the prophylaxis and treatment of such infections and diseases, as well as pharmaceutical compositions which can be used for the prophylaxis and/or treatment of Hepatitis C Virus infections and diseases.

This object is solved by the teaching of the independent claims. Further advantageous features, aspects and details of the invention are evident from the dependent claims, the description, the examples, and the drawings.

According to one aspect, the present invention refers to novel compounds having the general formula (I): 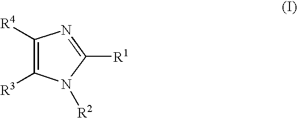
wherein:
R¹, R^{1′}, and R^{1″} represent independently of each other 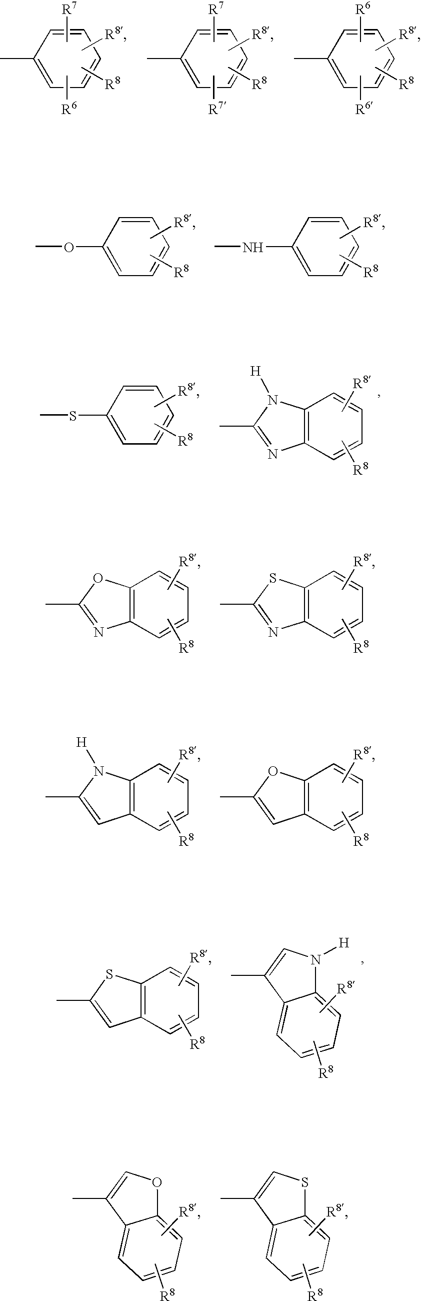 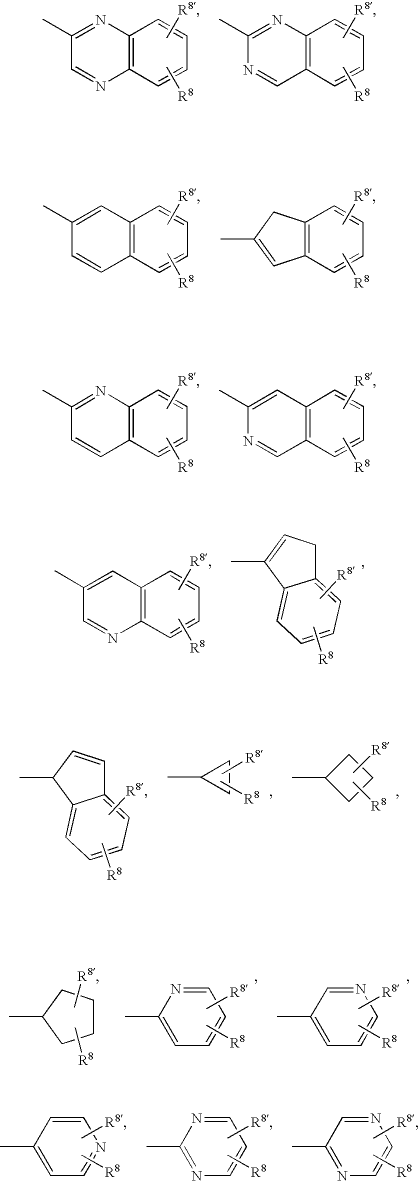 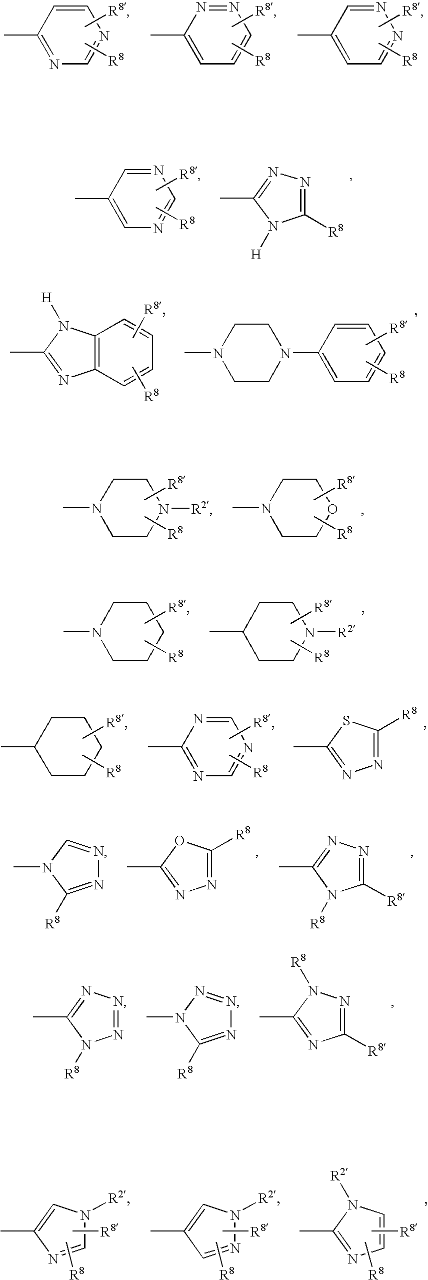 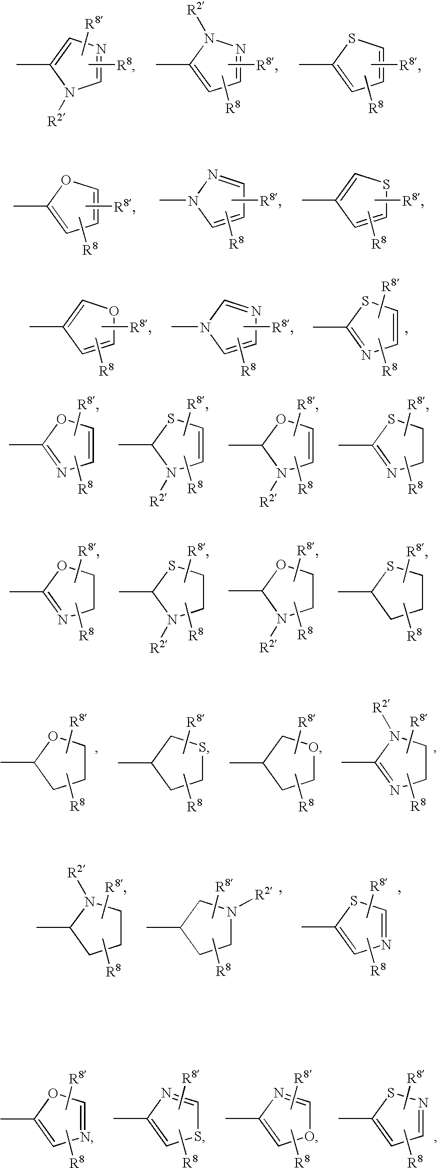 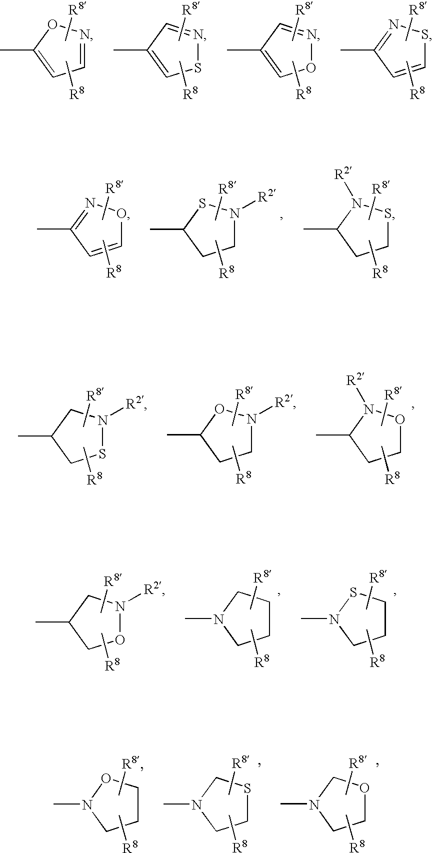
R², R^{2′}, and R^{2″} represent independently of each other —H, —CH₃, —C₂H₅, —CH═CH₂, —C≡CH, —C₃H₇, -cyclo-C₃H₅, —CH(CH₃)₂, —CH₂—CH═CH₂, —C(CH₃)═CH₂, —CH═CH—CH₃, —C≡C—CH₃, —CH₂—C≡CH, —C₄H₉, -cyclo-C₄H₇, —CH—CH(CH₃)₂, —CH(CH₃)—C₂H₅, —C(CH₃)₃, —C₅H₁₁, -cyclo-C₅H₉, —C₆H₁₃, -cyclo-C₆H₁₁, -Ph, —C(R⁵)₃, —C(R^{5′})₃, —CR⁵(R^{5′})₂, CR⁵(R^{5′})R^{5″}, —C₂(R⁵)₅, —CH₂—C(R⁵)₃, —CH₂—C(R^{5′})₃, —CH₂—CR⁵(R^{5′})₂, —CH₂—CR⁵(R^{5′})R^{5″}, —C₃(R⁵)₇, —C₂H₄—C(R⁵)₃, —C₇H₁₅, -cyclo-C₇H₁₃, —CH₂Ph, —C₈H₁₇, -cyclo-C₈H₁₅, C₂H₄Ph, —CH═CH-Ph, —C≡C-Ph;
R³ and R⁴ represent independently of each other —R^{1′}, —R^{1″}, —R⁶, —R^{6′}, —OR^{2′}, —OR^{2″}, —SR^{2′}, —SR^{2″};
R⁵, R^{5′} and R^{5″} represent independently of each other —F, —Cl, —Br, —I, —CN;
R⁶ and R^{6′} represent independently of each other —R^{2′}, —R^{2″}, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C≡C—CH₃, —C≡C—CH═CH₂, —CH═CH—C≡CH, —C≡C—C≡CH, —C₂H₄—CH(CH₃)₂, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₃H₆—CH═CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, —C(CH₃)═C(CH₃)₂, —C₃H₆—C≡CH, —C≡C—C₃H₇, —C₂H₄—C≡C—CH₃, —CH₂—C≡C—C₂H₅, —CH₂—C≡C—CH═CH₂, —CH₂—CH═CH—C≡CH, —CH₂—C≡C—C≡CH, —C≡C—CH═CH—CH₃, —CH═CH—C≡C—CH₃, —C≡C—C≡C—CH₃, —C≡C—CH₂—CH═CH₂, —CH═CH—CH₂—C≡CH, —C≡C—CH₂—C≡CH, —C(CH₃)═CH—CH═CH₂, —CH═C(CH₃)—CH═CH₂, —CH═CH—C(CH₃)═CH₂, —C(CH₃)═CH—C≡CH, —CH═C(CH₃)—C≡CH, —C≡C—C(CH₃)═CH₂, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH═CH₂, —CH═CH—C₄H₉, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C₄H₈—C≡CH, —C≡C—C₄H₉, —C₃H₆—C≡C—CH₃, —CH₂—C≡C—C₃H₇, —C₂H₄—C≡C—C₂H₅, -o-C₆H₄—R², -o-C₆H₄—R^{2′}, -m-C₆H₄—R², -m-C₆H₄—R^{2′}, -p-C₆H₄—R², -p-C₆H₄—R^{2′}, -o-CH₂—C₆H₄—R², -o-CH₂—C₆H₄—R^{2′}, -m-CH₂—C₆H₄—R², -m-CH₂—C₆H₄—R^{2′}, -p-CH₂—C₆H₄—R², -p-CH₂—C₆H₄—R^{2′};
R⁷ and R^{7′} represent independently of each other —R^{5′}, —R^{5″}, —H, —NO₂, —NO, —N₃, —OCN, —NCO, —SCN, —NCS, —COCN, —COOR^{2′}, —COOR^{2″}, —CO—R^{2′}, —CO—R^{2″}, —CONR^{2′}R^{2″}, —NR^{2′}R^{2″}, —NR⁶R^{6′}, —N^{⊕}R^{2′}R^{2″}R^{6′}, —SOR^{2′}, —SOR^{2″}, —SO₂R^{2′}, —SO₂R^{2″}, —SO₃R^{2′}, —SO₃R^{2″}, —NHCO—R^{2′}, —NHCO—R^{2″}, —NHCOO—R^{2′}, —NHCOO—R^{2″}, —OCONR^{2′}R^{2″}, —OCONR⁶R^{6′}, OCOR^{2′}, —OCOR^{2″}, —NH—SO₂—R^{2′}, —NH—SO₂—R^{2″}, —SO₂—NR^{2′}R^{2″}, —SO₂—NR⁶R^{6′}, —NH—CO—NH—R^{2′}, —NH—CO—NH—R^{2″}, —NH—CS—NH—R^{2′}, —NH—CS—NH—R^{2″}, —OR^{2′}, OR^{2″}, —SR^{2′}, —SR^{2″};
R⁸ and R^{8′} represent independently of each other —R⁷, —R^{7′}, —R^{6′}, —R^{6″};
   and pharmaceutically acceptable salts thereof.

The above-mentioned imidazole compounds according to general formula (I) as well as their pharmaceutically acceptable salts are particularly useful as agents against Hepatitis C Virus infections and diseases associated with such infections.

According to a preferred embodiment, in the general formula (I) R¹, R^{1′}, and R^{1″} represent independently of each other 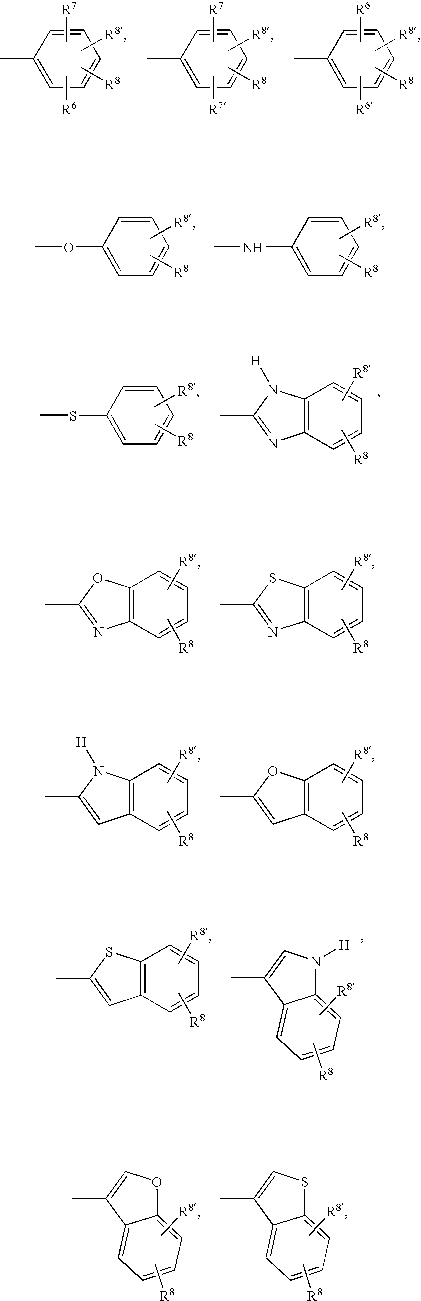 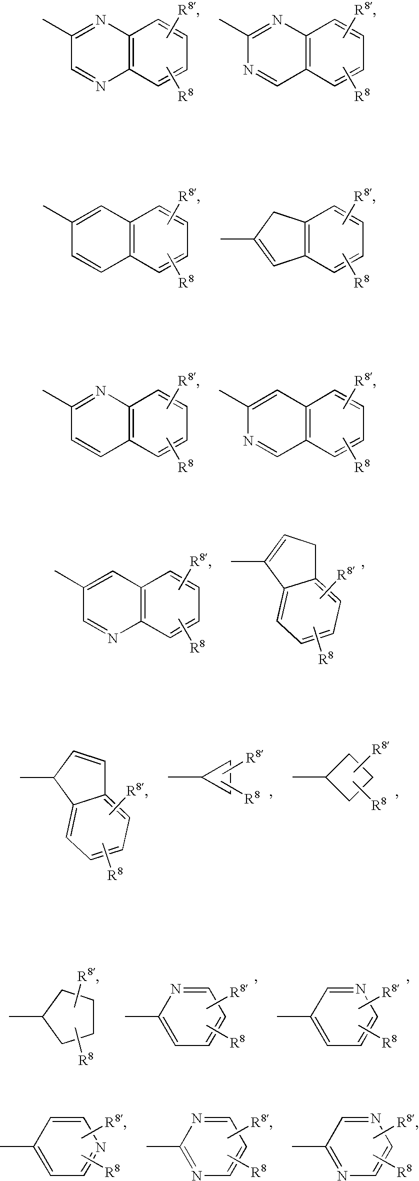 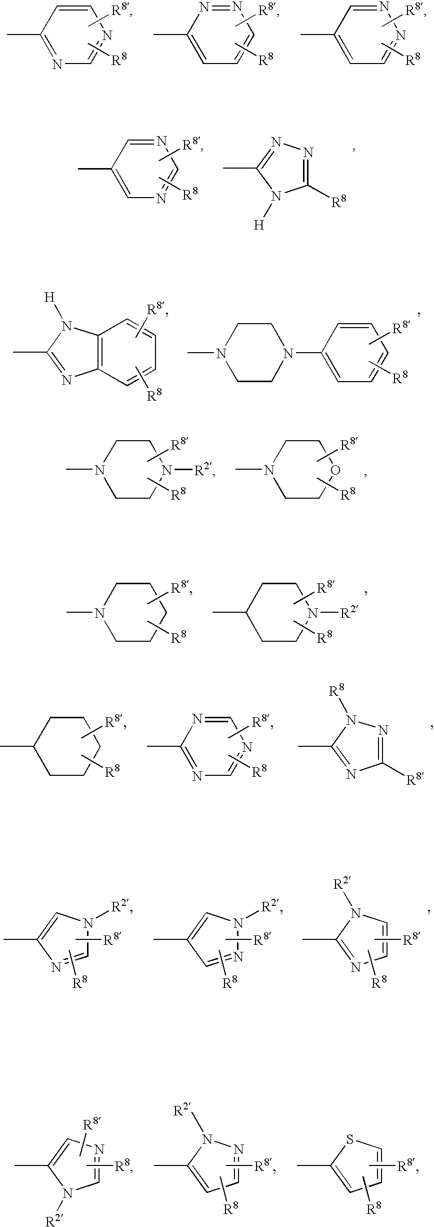 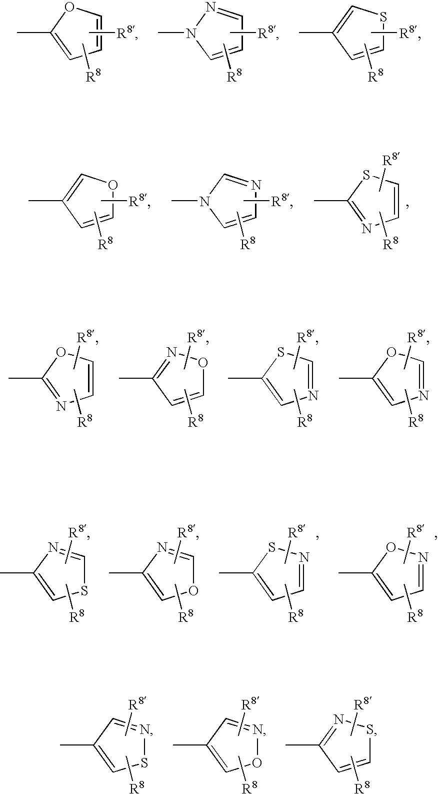
and R^{2′}, R⁶, R^{6′}, R⁷, R^{7′}, R⁸, R^{8′} have the meanings as defined above.

Specifically, R¹, R^{1′}, and R^{1″} represent independently of each other 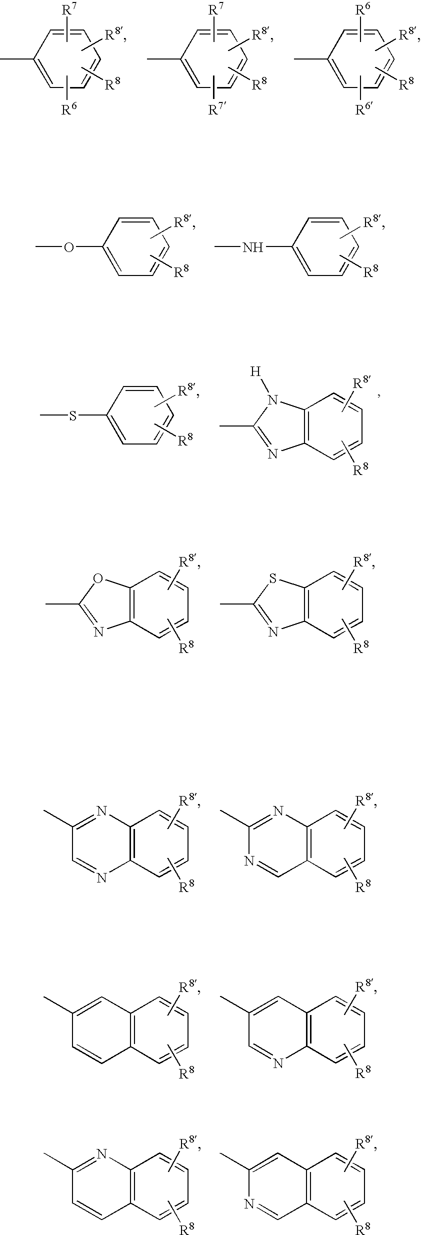 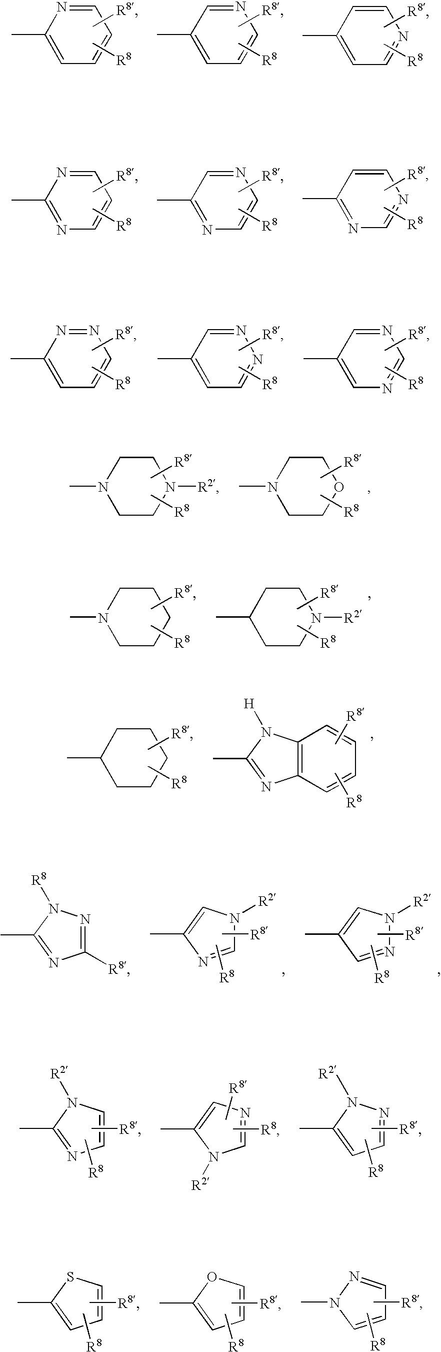 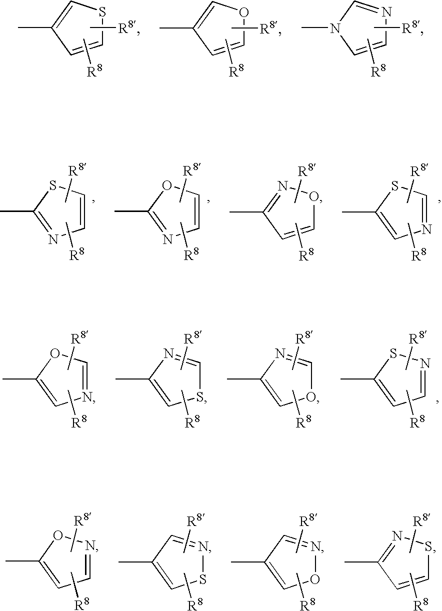
and R^{2′}, R⁶, R^{6′}, R⁷, R^{7′}, R⁸, R^{8′} have the meanings as defined above.

Even more specifically, R¹, R^{1′}, and R^{1″} represent independently of each other 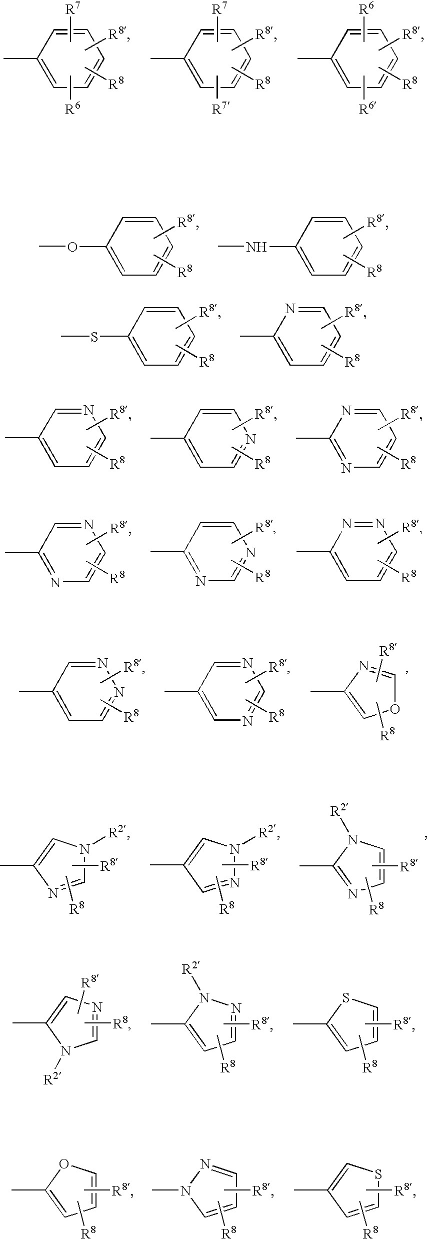 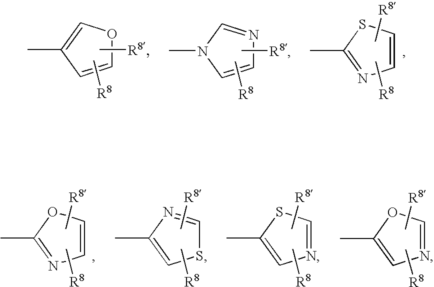
and R^{2′}, R⁶, R^{6′}, R⁷, R^{7′}, R⁸, R^{8′} have the meanings as defined above.

According to a further preferred embodiment R², R^{2′}, and R^{2″} represent independently of each other —H, —CH₃, —C₂H₅, —CH═CH₂, —C≡CH, —C₃H₇, -cyclo-C₃H₅, —CH(CH₃)₂, —CH₂—CH═CH₂, —C₄H₉, -cyclo-C₄H₇, —CH₂—CH(CH₃)₂, —CH(CH₃)—C₂H₅, —C(CH₃)₃, —C₅H₁₁, -cyclo-C₅H₉, —C₆H₁₃, -cyclo-C₆H₁₁, -Ph, —C(R⁵)₃, —C(R^{5′})₃, —CR⁵(R^{5′})₂, —CH₂Ph; and R⁵, R^{5′} have the meanings as defined above.

In addition, preferred are compounds wherein R⁵, R^{5′} and R^{5″} represent independently of each other —F, —Cl, —Br.

Furthermore, preferred are compounds wherein R⁶ and R^{6′} represent independently of each other —R^{2′}, —R^{2″}, -o-C₆H₄—R², -o-C₆H₄—R^{2′}, -m-C₆H₄—R², -m-C₆H₄—R^{2′}, -p-C₆H₄—R², -p-C₆H₄—R^{2′}, -o-CH₂—C₆H₄—R², -o-CH₂—C₆H₄—R^{2′}, -m-CH₂—C₆H₄—R², -m-CH₂—C₆H₄—R^{2′}, -p-CH₂—C₆H₄—R², -p-CH₂—C₆H₄—R^{2′}; and R², R^{2′}, R^{2″} have the meanings as defined above.

Among those compounds, R⁶ and R^{6′} specifically represent independently of each other —H, —CH₃, —C₂H₅, —CH═CH₂, —C≡CH, —C₃H₇, -cyclo-C₃H₅, —CH(CH₃)₂, —CH₂—CH═CH₂, —C₄H₉, -cyclo-C₄H₇, —CH₂—CH(CH₃)₂, —CH(CH₃)—C₂H₅, —C(CH₃)₃, —C₅H₁₁, -cyclo-C₅H₉, —C₆H₁₃, -cyclo-C₆H₁₁, -Ph, —C(R⁵)₃, —C(R^{5′})₃, —CR⁵(R^{5′})₂, —CH₂Ph, -o-C₆H₄—CH₃, -o-C₆H₄—C₂H₅, -m-C₆H₄—CH₃, -m-C₆H₄—C₂H₅, -p-C₆H₄—CH₃, -p-C₆H₄—C₂H₅, -o-CH₂—C₆H₄—CH₃, -o-CH₂—C₆H₄—C₂H₅, -m-CH₂—C₆H₄—CH₃, -m-CH₂—C₆H₄—C₂H₅, -p-CH₂—C₆H₄—CH₃, -p-CH₂—C₆H₄—C₂H₅; and R⁵, R^{5′} have the meanings as defined above.

According to further preferred embodiment of formula (I), R⁷ and R^{7′} represent independently of each other —F, —Cl, —Br, —H, —NO₂, —COOR^{2′}, —COOR^{2″}, —CO—R^{2′}, —CO—R^{2″}, —CONR^{2′}R^{2″}, —NR^{2′}R^{2″}, —NR⁶R^{6′}, —SOR^{2′}, —SOR^{2″}, —SO₂R^{2′}, —SO₂R^{2″}, —SO₃R^{2′}, —SO₃R^{2″}, —NHCO—R^{2′}, —NHCO—R^{2″}, —OCOR^{2′}, —OCOR^{2″}, —OR^{2′}, —OR^{2″}, —SR^{2′}, —SR^{2″}; and R^{2′}, R^{2″}, R⁶, R^{6′} have the meanings as defined above.

Preferred compounds falling under the general formula (I) above are
(Compound 1) 4-[5-(4-Fluoro-phenyl)-2-(4-isopropyl-phenyl)-3H-imidazole-4-yl]-pyridine,
(Compound 2) 3-[4-(4-Fluoro-phenyl)-5-pyridine-4-yl-1H-imidazole-2-yl]-4-nitro-phenol,
(Compound 3) 4-[4-(4-Fluoro-phenyl)-5-pyridine-4-yl-1H-imidazole-2-yl]-2-nitro-phenol,
(Compound 4) 4-[5-(4-Fluoro-phenyl)-2-(3-trifluoromethyl-phenyl)-3H-imidazole-4-yl]-pyridine,
(Compound 5) 2,6-Di-tert-butyl-4-[4-(4-fluoro-phenyl)-5-pyridine-4-yl)-1H-imidazole-2-yl]-phenol,
(Compound 6) 4-[2-(2,5-Bis-trifluoromethyl)-5-(4-fluoro-phenyl)-3H-imidazole-4-yl]-pyridine,
(Compound 7) 4-[5-(4-Fluoro-phenyl)-2-furan-2-yl-3H-imidazole-4-yl]-pyridine,
(Compound 8) 4-[5-(4-Fluoro-phenyl)-2-(2-methoxy-phenyl)-3H-imidazole-4-yl]-pyridine,
(Compound 9) 4-[5-(4-Fluoro-phenyl)-2-(5-methyl-furan-2-yl)-3H-imidazole-4-yl]-pyridine,
(Compound 10) 4-[5-(4-Fluoro-phenyl)-2-(3-methoxy-phenyl)-3H-imidazole-4-yl]-pyridine,
(Compound 11) 4-[5-(4-Fluoro-phenyl)-2-p-tolyl-3H-imidazole-4-yl]-pyridine,
(Compound 12) 4-[5-(4-Fluoro-phenyl)-2-(4-methoxy-phenyl)-3H-imidazole-4-yl]-pyridine,
(Compound 13) 4-[5-(4-Fluoro-phenyl)-2-(2-chloro-phenyl)-3H-imidazole-4-yl]-pyridine,
(Compound 14) 4-[5-(4-Fluoro-phenyl)-2-(2,4,6-trimethyl-phenyl)-3H-imidazole-4-yl]-pyridine,
(Compound 15) 4-[5-(4-Fluoro-phenyl)-2-(2,4-dichloro-phenyl)-3H-imidazole-4-yl]-pyridine,
(Compound 16) 4-[5-(4-Fluoro-phenyl)-2-(2,3-dichloro-phenyl)-3H-imidazole-4-yl]-pyridine,
(Compound 17) 4-[4-(4-Fluoro-phenyl)-5-pyridine-4-yl)-1H-imidazole-2-yl]-2-methoxy-phenol,
(Compound 18) 4-[5-(4-Fluoro-phenyl)-2-(2-nitro-phenyl)-3H-imidazole-4-yl]-pyridine,
(Compound 19) 4-[4-(4-Fluoro-phenyl)-5-pyridine-4-yl-1H-imidazole-2-yl]-benzene-1,2-diol,
(Compound 20) 4-[4-(4-Fluoro-phenyl)-5-pyridine-4-yl-1H-imidazole-2-yl]-phenol,
(Compound 21) 4-[2-(4,5-Dimethoxy-2-nitro-phenyl)-5-(4-fluoro-phenyl)-3H-imidazole-4-yl]-pyridine,
(Compound 22) 4-[5-(4-Fluoro-phenyl)-2-(3-chloro-phenyl)-3H-imidazole-4-yl]-pyridine,
(Compound 23) 4-[5-(4-Fluoro-phenyl)-2-(3-bromo-phenyl)-3H-imidazole-4-yl]-pyridine,
(Compound 24) 4-[5-(4-Fluoro-phenyl)-2-(3-nitro-phenyl)-3H-imidazole-4-yl]-pyridine,
(Compound 25) 4-[5-(4-Fluoro-phenyl)-2-(4-nitro-phenyl)-3H-imidazole-4-yl]-pyridine,
(Compound 26) 4-[5-(4-Fluoro-phenyl)-2-naphtalene-1-yl-3H-imidazole-4-yl]-pyridine,
(Compound 27) 4-[2-(3,5-Bis-trifluoromethyl-phenyl)-5-(4-fluoro-phenyl)-3H-imidazole-4-yl]-pyridine,
(Compound 28) {4-[4-(4-Fluoro-phenyl)-5-pyridine-4-yl-1H-imidazole-2-yl]-phenyl}-dimethyl-amine
(Compound 29) 4-[5-(4-Fluoro-phenyl)-2-(3,4-dichloro-phenyl)-3H-imidazole-4-yl]-pyridine,
(Compound 30) 4-[5-(4-Fluoro-phenyl)-2-(4-trifluoromethyl-phenyl)-3H-imidazole-4-yl]-pyridine,
(Compound 31) 4-[4-(4-Fluoro-phenyl)-5-pyridine-4-yl-1H-imidazole-2-yl]-2,6-dimethyl-phenol,
(Compound 32) 4-[5-(4-Fluoro-phenyl)-2-(4-methylsulfanyl-phenyl)-3H-imidazole-4-yl]-pyridine,
(Compound 33) 3-[4-(4-Fluoro-phenyl)-5-pyridine-4-yl-1H-imidazole-2-yl]-1H-indole,
(Compound 34) 4-[5-(4-Fluoro-phenyl)-2-(4-chloro-phenyl)-3H-imidazole-4-yl]-pyridine,
(Compound 35) 4-[5-(4-Fluoro-phenyl)-2-thiophene-2-yl-3H-imidazole-4-yl]-pyridine
(Compound 36) 4-[5-(4-Fluoro-phenyl)-2-(4-bromo-phenyl)-3H-imidazole-4-yl]-pyridine,
(Compound 37) 4-[2-(3,4-Dimethoxy-phenyl)-5-(4-fluoro-phenyl)-3H-imidazole-4-yl]-pyridine,
(Compound 38) 4-[5-(4-Fluoro-phenyl)-2-(4-methanesulfinyl-phenyl)-3H-imidazole-4-yl]-pyridine,
(Compound 39) 4-[5-(3-Iodo-phenyl)-2-(4-methanesulfinyl-phenyl)-3H-imidazole-4-yl]-pyridine,
(Compound 40) 6-(4-Fluoro-phenyl)-5-pyridine-4-yl-3,7-dihydro-2H-imidazole-[2,1-b]thiazole,
(Compound 41) 4-[5-Ethyl-2-(4-methoxy-phenyl)-1H-imidazole-4-yl]-pyridine,
(Compound 42) 4-[2,5-Bis-(4-chloro-phenyl)-1H-imidazole-4-yl]-pyridine,
(Compound 43) 4-[2-(4-Bromo-phenyl)-5-(4-chloro-phenyl)-1H-imidazole-4-yl]-pyridine,
(Compound 44) 4-[2-(2-Chloro-phenyl)-5-(4-chloro-phenyl)-1H-imidazole-4-yl]-pyridine,
(Compound 45) 4-[2-(3-Bromo-phenyl)-5-(4-chloro-phenyl)-1H-imidazole-4-yl]-pyridine,
(Compound 46) 4-[5-(4-Chloro-phenyl)-2-(2,3-dichloro-phenyl)-1H-imidazole-4-yl]-pyridine,
(Compound 47) 3-[5-(4-Chloro-phenyl)-4-pyridine-4-yl-1H-imidazole-2-yl]-4-nitro-phenol,
(Compound 48) 4-[5-(4-Chloro-phenyl)-2-(4-fluoro-phenyl)-1H-imidazole-4-yl]-pyridine,
(Compound 49) 4-[5-(4-Chloro-phenyl)-2-naphtalene-1-yl-1H-imidazole-4-yl]-pyridine,
(Compound 50) 4-[2-(3-Chloro-phenyl)-5-(4-chloro-phenyl)-1H-imidazole-4-yl]-pyridine,
(Compound 51) 4-[5-(4-Chloro-phenyl)-2-(3-methoxy-phenyl)-1H-imidazole-4-yl]-pyridine,
(Compound 52) 4-[5-(4-Chloro-phenyl)-2-(2-methoxy-phenyl)-1H-imidazole-4-yl]-pyridine,
(Compound 53) 4-[5-(4-Chloro-phenyl)-4-pyridine-4-yl-1H-imidazole-2-yl]-benzene-1,3-diol,
(Compound 54) 4-[5-(4-Chloro-phenyl)-4-pyridine-4-yl-1H-imidazole-2-yl]-2-methoxy-phenol,
(Compound 55) 4-[2-(3-Bromo-phenyl)-5-(3-trifluoromethyl-phenyl)-1H-imidazole-4-yl]-pyridine,
(Compound 56) 4-[2-(4-Trifluoromethyl-phenyl)-5-(3-trifluoromethyl-phenyl)-1H-imidazole-4-yl]-pyridine,
(Compound 57) 4-[2-(4-Bromo-phenyl)-5-(3-trifluoromethyl-phenyl)-1H-imidazole-4-yl]-pyridine,
(Compound 58) 4-[5-(3-Iodo-phenyl)-2-(4-trifluoromethyl-phenyl)-1H-imidazole-4-yl]-pyridine,
(Compound 59) 4-[5-(4-Chloro-phenyl)-2-(4-isopropyl-phenyl)-1H-imidazole-4-yl]-pyridine,
(Compound 60) 4-[5-(4-Chloro-phenyl)-4-pyridine-4-yl-1H-imidazole-2-yl]-2,6-dimethyl-phenol,
(Compound 61) 4-[5-(4-Chloro-phenyl)-2-(2,4-Dichloro phenyl)-1H-imidazole-4-yl]-pyridine,
(Compound 62) 4-[5-(4-Chloro-phenyl)-4-pyridine-4-yl-1H-imidazole-2-yl]-benzonitrile,
(Compound 63) 4-[5-(4-Chloro-phenyl)-4-pyridine-4-yl-1H-imidazole-2-yl]-phenol,
(Compound 64) 2,6-Di-tert-butyl-4-[5-(4-chloro-phenyl)-4-pyridine-4-yl-1H-imidazole-2-yl]-phenol
(Compound 65) 4-[5-(4-Chloro-phenyl)-2-(3,4-dimethoxy-phenyl)-1H-imidazole-4-yl]-pyridine,
(Compound 66) 4-[5-(4-Chloro-phenyl)-2-(3-nitro-phenyl)-1H-imidazole-4-yl]-pyridine,
(Compound 67) 4-[5-(4-Chloro-phenyl)-2-(3,4-Dichloro phenyl)-1H-imidazole-4-yl]-pyridine,
(Compound 68) 4-[5-(4-Chloro-phenyl)-2-(4-methoxy-phenyl)-1H-imidazole-4-yl]-pyridine,
(Compound 69) 4-[5-(4-Chloro-phenyl)-4-pyridine-4-yl-1H-imidazole-2-yl]-2,6-diisopropyl-phenol,
(Compound 70) N-{4-[5-(4-Chloro-phenyl)-4-pyridine-4-yl-1H-imidazole-2-yl]-acetamide,
(Compound 71) 4-[2-(3,4-Dichloro-phenyl)-5-(3-trifluoromethyl-phenyl)-1H-imidazole-4-yl]-pyridine,
(Compound 72) 4-[2-(4-Chloro-phenyl)-5-(3-trifluoromethyl-phenyl)-1H-imidazole-4-yl]-pyridine,
(Compound 73) 4-[4-Pyridine-4-yl-5-(3-trifluoromethyl-phenyl)-1H-imidazole-2-yl]-phenol,
(Compound 74) 4-[4-Pyridine-4-yl-5-(3-trifluoromethyl-phenyl)-1H-imidazole-2-yl]-2-methoxy-phenol,
(Compound 75) 4-[2-(3-Chloro-phenyl)-5-(3-trifluoromethyl-phenyl)-1H-imidazole-4-yl]-pyridine,
(Compound 76) 4-[2-(4-Methylsulfanyl-phenyl)-5-(3-trifluoromethyl-phenyl)-1H-imidazole-4-yl]-pyridine,
(Compound 77) 3-[4-Pyridine-4-yl-5-(3-trifluoromethyl-phenyl)-1H-imidazole-2-yl]-phenol,
(Compound 78) 4-[2-(3-Bromo-phenyl)-5-(3-iodo-phenyl)-1H-imidazole-4-yl]-pyridine,
(Compound 79) 4-[5-(3-Iodo-phenyl)-4-pyridin-4-yl-1H-imidazole-2-yl]-2,6-dimethyl-phenol,
(Compound 80) 4-[2-(4-Bromo-phenyl)-5-(3-iodo-phenyl)-1H-imidazole-4-yl]-pyridine,
(Compound 81) 4-[2-(3-Chloro-phenyl)-5-(3-iodo-phenyl)-1H-imidazole-4-yl]-pyridine,
(Compound 82) 4-[2-(4-Fluoro-phenyl)-5-(3-iodo-phenyl)-1H-imidazole-4-yl]-pyridine,
(Compound 83) 4-[2-Naphtalene-1-yl-5-phenyl-1H-imidazole-4-yl]-pyridine,
(Compound 84) 4-(5-Phenyl-2-styryl-1H-imidazole-4-yl]-pyridine,
(Compound 85) 4-[5-Phenyl-2-(4-trifluoromethyl-phenyl)-1H-imidazole-4-yl]-pyridine,
(Compound 86) 2-Nitro-4-(5-phenyl-4-pyridine-4-yl-1H-imidazole-2-yl)-phenol,
(Compound 87) 4-[2-(3-Bromo-phenyl)-5-phenyl-1H-imidazole-4-yl]-pyridine,
(Compound 88) 2,6-Dimethyl(5-phenyl-4-pyridine-4-yl-1H-imidazole-2-yl)-phenol,
(Compound 89) 4-[2-(3,4-Bis-benzyloxy-phenyl)-5-phenyl-1H-imidazole-4-yl]-pyridine,
(Compound 90) 4-[2-(3,4-Dimethoxy-phenyl)-5-phenyl-1H-imidazole-4-yl]-pyridine,
(Compound 91) 4-[2-(3-Nitro-phenyl)-5-phenyl-1H-imidazole-4-yl]-pyridine,
(Compound 92) 4-[2-(4-Chloro-phenyl)-5-phenyl-1H-imidazole-4-yl]-pyridine,
(Compound 93) 2-(5-Phenyl-4-pyridine-4-yl-1H-imidazole-2-yl)-benzene-1,4-diol,
(Compound 94) 4-(5-Phenyl-4-pyridine-4-yl-1H-imidazole-2-yl)-phenol,
(Compound 95) 3-(5-Phenyl-4-pyridine-4-yl-1H-imidazole-2-yl)-phenol,
(Compound 96) 4-[2-(4-Bromo-phenyl)-5-phenyl-1H-imidazole-4-yl]-pyridine,
(Compound 97) 2-Methoxy-4-(5-phenyl-4-pyridine-4-yl-1H-imidazole-2-yl)-phenol,
(Compound 98) 4-[2-(4-Isopropyl-phenyl)-5-phenyl-1H-imidazole-4-yl]-pyridine,
(Compound 99) 4-[2-(2,3-Dichloro-phenyl)-5-phenyl-1H-imidazole-4-yl]-pyridine,
(Compound 100) 4-[2-(2,4-Dichloro-phenyl)-5-phenyl-1H-imidazole-4-yl]-pyridine,
(Compound 101) 4-[2-(4-Methylsulfanyl-phenyl)-5-phenyl-1H-imidazole-4-yl]-pyridine,
(Compound 102) 4-[2-(2-Chloro-phenyl)-5-phenyl-1H-imidazole-4-yl]-pyridine,
(Compound 103) 4-[2-(4-Methoxy-phenyl)-5-phenyl-1H-imidazole-4-yl]-pyridine,
(Compound 104) 4-[2-(3-Methoxy-phenyl)-5-phenyl-1H-imidazole-4-yl]-pyridine,
(Compound 105) 4-[2-(2-Methoxy-phenyl)-5-phenyl-1H-imidazole-4-yl]-pyridine,
(Compound 106) 4-[2-(3-Chloro-phenyl)-5-phenyl-1H-imidazole-4-yl]-pyridine,
(Compound 107) 2,6-Di-tert-butyl-4-(5-phenyl-4-pyridine-4-yl-1H-imidazole-2-yl)phenol,
(Compound 108) 4-(5-Phenyl-4-pyridine-4-yl-1H-imidazole-2-yl)-benzonitrile,
(Compound 109) N-[4-(5-Phenyl-4-pyridine-4-yl -1H-imidazole-2-yl)-phenyl]-acetamide,
(Compound 110) 4-{2-[2-(2-Methoxy-phenyl)-vinyl]-5-phenyl-1H-imidazole-4-yl}-pyridine,
(Compound 111) 4-[5-(3-Iodo-phenyl)₄-pyridine-4-yl-1H-imidazole-2-yl]-phenol,
(Compound 112) 4-[2-(2,3-Dichloro-phenyl)-5-(3-iodo-phenyl)-1H-imidazole-4-yl]-pyridine,
(Compound 113) 4-[5-(4-Chloro-phenyl)-2-(4-methylsulfanyl-phenyl)-1H-imidazole-4-yl]-pyridine,
(Compound 114) 4-[5-(4-Chloro-phenyl)₄-pyridine-4-yl-1H-imidazole-2-yl]-dimethyl amine,
(Compound 115) 4-[5-(3-Iodo-phenyl)-2-(5-methyl-furan-2-yl)-1H-imidazole-4-yl]-pyridine,
(Compound 116) 4-[4-(4-Fluoro-phenyl)-5-pyridine-4-yl-1H-imidazole-2-yl]-benzylamine,
(Compound 117) 4-[5-(3-Iodo-phenyl)-2-(4-methylsulfanylphenyl)-1H-imidazole-4-yl]-pyridine,
(Compound 118) 4-[2-(4-Methanesulfinyl-phenyl)-5-phenyl-3H-imidazole-4-yl]-pyridine,
(Compound 119) 4-[5-(4-Fluoro-phenyl)-4-pyridin-4-yl-3H-imidazole-2-yl]-phenylamine,
(Compound 120) {4-[5-(3-Iodo-phenyl)₄-pyridin-4-yl-1H-imidazole-2-yl]-phenyl}-methanol,
(Compound 121) 4-[5-(4-Fluoro-phenyl)-4-pyridin-4-yl-1H-imidazole-2-yl]-benzylamine,
(Compound 122) 2-(3,4-Dimethoxyphenyl)-4,5-bis-(4-methoxyphenyl)-1H-imidazole,
(Compound 123) 4-[4,5-Bis-(4-methoxyphenyl)-1H-imidazole-2-yl]-2,6-bis-tert-butylphenol,
(Compound 124) 4-[4,5-Bis-(4-methoxyphenyl)-1H-imidazole-2-yl]-2-methoxy-phenol,
(Compound 125) 4-[4,5-Bis-(4-bromophenyl)-1H-imidazole-2-yl]-2-methoxy-phenol,
(Compound 126) 4-[4,5-Bis-(4-methoxyphenyl)-2-styryl-1H-imidazole,
(Compound 127) 4-[4,5-Bis-(4-methoxyphenyl)-2-(4-trifluoromethyl-phenyl)-1H-imidazole],
(Compound 128) 4-[4,5-Bis-(4-methoxyphenyl)-2-(3-trifluoromethyl-phenyl)-1H-imidazole,
(Compound 129) 4-[4,5-Bis-(4-methoxyphenyl)-1H-imidazole-2-yl]-2-nitro-phenol,
(Compound 130) 4-[4,5-Bis-(4-methoxyphenyl)-1H-imidazole-2-yl]-4-nitro-phenol,
(Compound 131) 4-[4,5-Bis-(4-methoxyphenyl)-1H-imidazole-2-yl]-phenol,
(Compound 132) 2-(3-Bromo-phenyl)-4,5-bis-(4-methoxyphenyl)-1H-imidazole,
(Compound 132) 2-(3,4-Diphenoxy-phenyl)-4,5-bis-(4-methoxyphenyl)-1H-imidazole,
(Compound 133) {4-[4,5-Bis-(4-methoxyphenyl)-1H-imidazole-2-yl]-phenyl}-dimethylamine,
(Compound 134) 2-(4-Chloro-phenyl)-4,5-bis-(4-methoxyphenyl)-1H-imidazole,
(Compound 135) 2-(4-Bromo-phenyl)-4,5-bis-(4-methoxyphenyl)-1H-imidazole,
(Compound 136) 4,5-Bis-(4-methoxyphenyl)-2-(3-nitrophenyl)-1H-imidazole,
(Compound 137) 4,5-Bis-(4-methoxyphenyl)-2-naphthalen-1-yl-1H-imidazole,
(Compound 138) 2-(2,3-Dichlorophenyl)-4,5-bis-(4-methoxyphenyl)-1H-imidazole,
(Compound 139) 2-(2,4-Dichlorophenyl)-4,5-bis-(4-methoxyphenyl)-1H-imidazole,
(Compound 140) 4,5-Bis-(4-methoxyphenyl)-2-(4-nitro-phenyl)-H-imidazole,
(Compound 141) 4-[4,5-Bis-(4-methoxyphenyl)-1H-imidazole-2-yl]-benzene-1,2-diol,
(Compound 142) 2-(4-Methoxy-3,5-dimethyl-phenyl)-4,5-bis-(4-methoxy-phenyl)-1H-imidazole,
(Compound 143) 4-[4,5-Bis-(4-methoxyphenyl)-1H-imidazole-2-yl]-1H-indole,
(Compound 144) 2-(3,4-Bis-benzyloxy-phenyl)-4,5-bis-(4-bromo-phenyl)-1H-imidazole,
(Compound 145) 4,5-Bis-(4-bromo-phenyl)-2-(4-isopropyl-phenyl)-1H-imidazole,
(Compound 146) 4,5-Bis-(4-bromo-phenyl)-2-(2,4-dichloro-phenyl)-1H-imidazole,
(Compound 147) 4,5-Bis-(4-bromo-phenyl)-2-(4-chloro-phenyl)-1H-imidazole,
(Compound 148) 4,5-Bis-(4-bromo-phenyl)-2-(4-trifluoromethyl-phenyl)-1H-imidazole,
(Compound 149) 4,5-Bis-(4-bromo-phenyl)-2-(3-trifluoromethyl-phenyl)-1H-imidazole,
(Compound 150) 2-(3,5-Bis-trifluoromethyl-phenyl)-4,5-bis-(4-bromo-phenyl)-1H-imidazole,
(Compound 151) 2-(3,5-Bis-trifluoromethyl-phenyl)-4,5-bis-(4-bromo-phenyl)-1H-imidazole,
(Compound 152) 4,5-Bis-(4-bromo-phenyl)-2-(3,4-dimethoxy-phenyl)-1H-imidazole,
(Compound 153) 4,5-Bis-(4-bromo-phenyl)-2-(4-methylsulfanyl-phenyl)-1H-imidazole,
(Compound 154) 2-(3-Bromo-phenyl)-4,5-bis-(4-bromo-phenyl)-1H-imidazole,
(Compound 155) 4,5-Bis-(4-bromo-phenyl)-2-(2,3-dichloro-phenyl)-1H-imidazole,
(Compound 156) 4,5-Bis-(4-bromo-phenyl)-2-(3-nitro-phenyl)-1H-imidazole,
(Compound 157) 4-[4,5-Bis-(4-bromo-phenyl)-1H-imidazole-2-yl]-2,6-dimethyl-phenol,
(Compound 158) 4,5-Bis-(4-bromo-phenyl)-2-(4,5-dimethoxy-2-nitro-phenyl)-1H-imidazole,
(Compound 159) 4-[4,5-Bis-(4-bromo-phenyl)-1H-imidazole-2-yl]-2-nitro-phenol,
(Compound 160) {4-[4,5-Bis-(4-bromo-phenyl)-1H-imidazole-2-yl]-phenyl}-dimethylamine,
(Compound 161) 4,5-Bis-(4-bromo-phenyl)-2-naphthalen-1-yl-1H-imidazole,
(Compound 162) 4,5-Bis-(4-bromo-phenyl)-2-(5-ethyl-furan-2-yl)-1H-imidazole,
(Compound 163) 4,5-Bis-(4-bromo-phenyl)-2-thiophen-2-yl-1H-imidazole,
(Compound 164) 3-[4,5-Bis-(4-bromophenyl)-1H-imidazole-2-yl]-1H-indole,
(Compound 165) 2-(3,4-Dimethoxy-phenyl)-4,5-di-thiophen-2-yl-1H-imidazole,
(Compound 166) 2-(4-Isopropyl-phenyl)-4,5-di-thiophen-2-yl-1H-imidazole,
(Compound 167) 2-(3-Bromo-phenyl)-4,5-di-thiophen-2-yl-1H-imidazole,
(Compound 168) 4,5-Di-thiophen-2-yl-2-(4-trifluoromethyl-phenyl)-1H-imidazole,
(Compound 169) 4,5-Di-thiophen-2-yl-2-(3-trifluoromethyl-phenyl)-1H-imidazole,
(Compound 170) [4-(4,5-Di-thiophen-2-yl-1H-imidazole-2-yl)-phenyl]-dimethylamine,
(Compound 171) 2-(3,4-Bis-benzyloxy-phenyl)-4,5-di-thiophen-2-yl-1H-imidazole,
(Compound 172) 2-Naphthalen-1-yl-4,5-di-thiophen-2-yl-1H-imidazole,
(Compound 173) 4-(4,5-Di-thiophen-2-yl-1H-imidazole-2-yl)-2-nitrophenol.

Especially preferred are the compounds 4-[4-(4-fluoro-phenyl)-5-pyridine-4-yl-1H-imidazole-2-yl]-phenol, 4-[5-(3-iodo-phenyl)-2-(4-methanesulfinyl-phenyl)-3H-imidazole-4-yl]-pyridine, or 4-[4-(4-fluoro-phenyl)-5-pyridine-4-yl-1H-imidazole-2-yl]-benzylamine falling under the general formula (I) above.

With regard to the synthesis and preparation of the imidazole derivatives of general formula (I) according to the present invention reference is made to the following documents.

Various imidazole compounds are known from DD 97654. Said patent of the German Democratic Republic was filed in 1972 and discloses methods for the synthesis of imidazole derivatives of the general formula (1). According to DD 97654 the imidazole derivatives of formula (I) can be synthesized by converting a suitable diketone with ammonia and a corresponding aldehyde or by converting an amide with ammonia Furthermore, according to DD 97654 the desired imidazole derivatives can be obtained by the reaction of an α-hydroxyketone with a suitable amidine or by subjecting an oxazole to an ammonia medium. Further methods for the synthesis of specific subgroups of imidazole derivatives are e.g. disclosed in EP-A-0712847. The imidazole derivatives of EP-A-0712847 are used as pesticides.

EP-A-0257897 discloses imidazole derivatives with a 2-phenyl or a substituted 2-phenyl group, methods for the preparation of these imidazole derivatives and pharmaceutical compositions comprising the same. According to EP-A-0257897, these imidazole compounds and the pharmaceutically acceptable salts thereof exhibit cardiotonic activity, anti-platelet activity and/or anti-inflammatory activity and are capable of reducing heart rate.

U.S. Pat. No. 5,656,644 discloses pyridyl imldazoles, processes for preparing these pyridyl imidazoles, the use thereof in the treatment of cytokine mediated diseases and compositions for use in such therapy. According to U.S. Pat. No. 5,656,644, said pyridyl imidazoles are used in association with the veterinary treatment of mammals, but not humans. In particular, said pyridyl imidazoles are used for therapeutical or prophylactical treatment of cytokine mediated diseases in animals.

The synthesis of 4,5-dithiophenyl-imidazole derivatives is described by K. Guven et al. (K. Guven et al., Bollettino Chimico Farmaceutica, 2002, 141(6), 443-446). According to this article, imidazole derivatives of general formula (I) can be synthesized by reacting di-(2-thienyl)ethandione and a suitable aromatic aldehyde in the presence of ammonium acetate in acetic acid. Either the classical reflux method or microwave irradiation method could by applied as alternative reaction conditions. It is stated in this article, that these compounds have antimicrobial activities.

Further pyridyl imidazoles are disclosed in U.S. Pat. No. 3,929,807. It is stated in this document that these pyridyl imidazoles have anti-inflammatory, antinociceptive and anti-pyretic activity and that these compounds are used as active ingredients of pharmaceutical compositions for the relief and removal of pain as well as for the treatment of rheumatic, arthritic and other inflammatory complaints.

Recent research has revealed how cells communicate with each other to coordinate the growth and maintenance of the multitude of tissues within the human body. A key element of this communication network is the transmission of a signal from the exterior of a cell to its nucleus, which results in the activation or suppression of specific genes. This process is called signal transduction.

An integral part of signal transduction is the interaction of ligands, their receptors and intracellular signal transduction molecules. Ligands are messengers that bind to specific receptors on the surface of target cells. As a result of the binding, the receptors trigger the activation of a cascade of downstream signaling molecules, thereby transmitting the message from the exterior of the cell to its nucleus. When the message reaches the nucleus, it initiates the modulation of specific genes, resulting in the production of RNA and finally proteins that carry out a specific biological function.

Disturbed activity of signal transduction molecules may lead to the malfunctioning of cells and disease processes. Specifically, interaction of HCV with host cells is necessary for the virus to replicate.

The antiviral therapeutic research approach described herein focuses on discovering the cellular signal transduction pathways involved in viral transfections. Identification of the signal transduction molecules that are key to viral infection provides for, among other things, novel targets for antiviral therapeutics, useful antiviral therapeutics, and new screening methods (e.g. assays) and materials to find and develop new antiviral agents.

In order to develop new pharmaceutically active compounds, a potential target for medical intervention has to be identified. Thus, processes for finding pharmaceutically effective compounds include target identification.

Target identification is basically the identification of a particular biological component, namely a protein and its association with particular disease states or regulatory systems. A protein identified in a search for a pharmaceutically active chemical compound (drug) that can affect a disease or its symptoms is called a target. Said target is involved in the regulation or control of biological systems and its function can be interfered with by a drug.

HCV is a member of the Flaviviridae family and harbours a plus strand RNA genome, which is translated into a single precursor polypeptide of about 3000 amino acids. Co- and posttranslational processing by both host cell and viral proteases generates the mature structural (C, E1, E2, p7) and non-structural (NS2, NS3, NS4A, NS5A and NS5B) HCV proteins. NS3, a viral component containing NTPase and RNA helicase activities, and the RNA-dependent RNA polymerase NS5B have been directly implicated in viral RNA replication. The NS5A protein is also assumed to play a role in viral replication, but its precise role remains to be defined. NS5A is phosphorylated in intact cells. It has been shown that casein kinase II (CKII) associates with the C-terminal portion of NS5A and phosphorylates it in vitro (Kim et. al., Bioch and Biophys Research Comm 257, 777-781, 1999). However host cell protein kinases mediating NS5A phosphorylation in vivo have not been identified yet. As phosphorylation of NS5A and its homologues is a conserved feature among different members of the Flaviviridae family, it appears likely that phosphorylation of NS5A plays an essential role during the HCV replication cycle (Reed et al., J. Virol. 72, 6199-6206, 1998). The cellular protein kinases involved, particularly the cellular kinases responsible for NS5A phosphorylation in vivo, could therefore serve as promising targets for antiviral therapeutic intervention.

CKI (Casein Kinase I) and CKII (Casein Kinase II) form part of a family of serine/threonine protein kinases that are present in all eukaryotes examined to date. CKI family members which include casein kinase I α, γ, ε and δ, have been implicated in the control of cytoplasmic and nuclear processes, including DNA replication and repair. CKII is usually expressed as a tetrameric complex with an αα′β₂, α₂β₂, or α′₂β₂ form, α and α′ being catalytic subunits and β being a regulatory subunit. The a catalytic subunit is activated by the regulatory subunit which undergoes autophosphorylation. The following articles refer to different Casein Kinases:
J. E. Allende et al., Journal of Cellular Biochemistry. 2002, 86(4), 805-814;
A. B. Tobin et al., Journal of Biological Chemistry 2000, 275(26), 19667-19675;
D. M. Virshup, Journal of Biological Chemistry, 1995, 270(25), 14875-14883;
Z. Yao et al., Neuroreport, 2000, 11(5), 951-955;
D. M. Virshup et al, Journal of Biological Chemistry 1998, 273(26), 15980-15984;

The approach to identify compounds with anti-HCV activity is focused on the identification and inhibition of cellular or HCV-specific signal transduction pathways and signal transduction enzymes critical for infectivity, replication, spread or pathogenicity of Hepatitis C Virus. Access to cellular system(s) that allow HCV infection or parts of the infectious cycle to take place within a cultured cell line is therefore crucial for the development of assays with selected compounds like known protein kinase inhibitors or compounds with kinase inhibitor-like structures.

The only reliable experimental HCV infection studies have been perfomed with chimpanzees. There is no simple cell culture infection system available for HCV. Although a number of reports has been published describing in vitro propagation attempts of HCV in primary cells and cell lines, questions remain concerning reproducibility, low levels of expression and properly controlled detection methods (reviewed in J. Gen Virol. 81, 1631; Antiviral Chemistry and Chemotherapy 10, 1999). The replicon system described by Bartenschlager and co-workers (Lohmann et al., Replication of subgenomic hepatitis C virus RNAs in a hepatoma cell line, Science 285, 110, 1999) was therefore investigated, which reproduces a crucial part of the HCV replication cycle, but does not lead to a productive infection or virus generation. Bartenschlager's group produced bicistronic recombinant RNAs, so-called “replicons”, which carry the Neomycin-phosphotransferase gene as well as a version of the HCV genome where the sequences for the structural HCV proteins were deleted. After transfection of the subgenomic HCV RNA molecules into the human hepatoma cell line Huh-7, cells supporting efficient RNA-dependent RNA replication of the HCV replicons were selected based on co-amplification of the neo gene and resulting resistance to the antibiotic G418. Integration of coding information into the cellular genome was an exclusion criterium for functional replicons. Several lines were established from G418 resistant clones with autonomously replicating HCV RNAs detectable by Northern blot. Minus-strand RNA replication intermediates were detected by Northern or metabolic radiolabelling, and the production of nonstructural HCV proteins was demonstrated by immunoprecipitation after metabolic labeling or Western blot.

Huh-pcDNA3 cells are Huh7 cells resistant to G418 by integration of a plasmid and serve as negative control.

The Huh-5-15 cell line with persistant replicon 1389/NS3-3′/wt, described in Lohmann et al., Science 285, 110-113, 1999, was used here as well as the Huh-5-2 cell line, which carries the persistant bicistronic replicon I389luc-ubi-neo/NS3-3′/5.1 that expresses a firefly luciferase-ubiquitin-neomycin phosphotransferase fusion protein.

According to a further aspect, the present invention relates to the human cellular kinases of the CKI family as targets for medical intervention against Hepatitis C Virus (HCV) infections. Furthermore, the present invention relates to a method for the detection of compounds for prophylaxis and/or treatment of HCV infections and diseases caused by Hepatitis C Virus infections, methods for treating infections and diseases induced by said HCV and to pharmaceutical compositions useful within said methods. Such selected compounds are particularly the imidazole compounds falling under the general formula (I) mentioned above as well as the preferred embodiments thereof, which selected compounds are effective for detection, prophylaxis and/or treatment of hepatitis C virus infections and diseases caused by hepatitis C virus infections. In addition thereto, the present invention describes solid supports useful for detecting hepatitis C virus infections and compounds suitable for preventing and/or treating hepatitis C virus infections and diseases caused by said HCV infections.

Based on the surprising results reported herein, one aspect of the present invention is directed to a screening method for the identification of compounds useful for prophylaxis and/or treatment of HCV infections and/or diseases. Specifically, this method involves contacting a test compound with the human cellular proteins casein kinase I α, δ, and ε and determining the activity of said human cellular proteins.

Disclosed herein is for the first time the role of human cellular casein kinases I α, δ, and ε in the signal transduction of the HCV infection process. As a result of these investigations, compounds and inhibitors against the above-mentioned casein kinases I α, δ, and ε may be found by using the inventive methods disclosed herein.

It is apparent to a person skilled in the art that detection includes any method known in the art useful to indicate the presence, absence, or amount of a detection target. Such methods may include, but are not limited to, any molecular or cellular techniques, used singularly or in combination, including, but not limited to: hybridization and/or binding techniques, including blotting techniques and immunoassays; labeling techniques (chemiluminescent, calorimetric, fluorescent, radioisotopic); spectroscopic techniques; separations technology, including precipitations, electrophoresis, chromatography, centrifugation, ultrafiltration, cell sorting; and enzymatic manipulations (e.g., digestion).

As used herein, the terms “HCV induced” or “HCV associated” diseases refer to the group of diseases comprising chronic hepatitis, liver cirrhosis and hepatocellular carcinoma.

Also described in the present invention are monoclonal or polyclonal antibodies which can bind to the human cellular proteins casein kinase I α, δ, and ε.

A further aspect of the present invention relates to a method for preventing and/or treating HCV infections and/or associated diseases in an individual comprising the step of administering a pharmaceutically effective amount of an agent which can inhibit at least partially the activity of the human cellular proteins casein kinase I α, δ, and ε, or which inhibits at least partially the production of the casein kinase I α, δ, and ε.

The term “individual” preferably refers to mammals, especially humans.

The methods disclosed herein can also be used for the treatment of hepatitis C virus strains resistant to medications currently available.

As used herein, the term “inhibitor” refers to any compound capable of downregulating, decreasing, suppressing or otherwise regulating the amount and/or activity of the human cellular proteins casein kinase I α, δ, and ε. Generally, said inhibitors, including suicide inhibitors, may be proteins, oligo- and polypeptides, nucleic acids, genes, chemical molecules, or other chemical moieties.

Especially preferred selected chemical compounds that are found to inhibit casein kinase I α, δ, and ε activity are imidazoles according to general formula (I) above, particularly compounds No. 1 to No. 116 recited above, and especially 4-[4-(4-fluoro-phenyl)-5-pyridine-4-yl-1H-imidazole-2-yl]-phenol, 4-[5-(3-iodo-phenyl)-2-(4-methanesulfinyl-phenyl)-3H-imidazole-4-yl]-pyridine, and 4-[4-(4-fluoro-phenyl)-5-pyridine-4-yl-1H-imidazole-2-yl]-benzylamine.

Another aspect of the present invention is directed to a method for regulating the production and/or replication of HCV in an individual comprising the step of administering an individual a pharmaceutically effective amount of an agent wherein said agent inhibits at least partially the activity of the human cellular proteins casein kinase I α, δ, and/or ε, or wherein said agent at least partially inhibits the production and/or replication of the human cellular proteins casein kinase I α, δ, and/or ε.

As used herein, the term “agent” refers to any compound, particularly chemical compound, capable of down- or upregulating, de- or increasing, suppressing, activating, stimulating or otherwise regulating the amount and/or activity of the human cellular proteins casein kinase I α, δ, and/or ε. Generally, said agents may be proteins, oligo- and polypeptides, nucleic acids, chemical molecules, or other chemical moieties.

A similar aspect relates to a method for regulating the production and/or replication of HCV in cells comprising the step of administering the cells a pharmaceutically effective amount of an agent wherein said agent inhibits at least partially the activity of at least one of the human cellular proteins casein kinase I α, δ, and ε, or wherein said agent at least partially inhibits the production of the human cellular proteins casein kinase I α, δ, and/or ε.

Monoclonal or polyclonal antibodies which have the capability to bind to the human cellular proteins casein kinase I α, δ, and/or ε may be used as effective agents within the above-mentioned methods.

As used herein, the term “regulating expression and/or activity” generally refers to any process that functions to control or modulate the quantity or activity (functionality) of a cellular component Static regulation maintains expression and/or activity at some given level. Upregulation refers to a relative increase in expression and/or activity. Accordingly downregulation refers to a relative decrease in expression and/or activity. Downregulation is synonymous with inhibition of a given cellular component's activity.

A further aspect of the invention is related to a method for regulating the expression of the human cellular proteins casein kinase I α, δ, and/or ε in an individual comprising the step of administering the individual a pharmaceutically effective amount of an agent wherein said agent inhibits at least partially the transcription of DNA or the translation of RNA encoding the human cellular proteins casein kinase I α, δ, and/or ε.

A still further aspect of the present invention relates to a method for regulating the expression of the human cellular proteins casein kinase I α, δ, and/or ε in cells comprising the step of administering the cells a pharmaceutically effective amount of an agent wherein said agent inhibits at least partially the transcription of DNA or the translation of RNA encoding the human cellular proteins casein kinase I α, δ, and/or ε.

According to still further aspect, the present invention relates to a method for detecting a Hepatitis C Virus infection and/or a disease associated therewith in an individual, the method comprising the following steps: a) providing a sample of the individual; and b) determining the activity, in the sample, of one or more proteins selected from the group consisting of casein kinase I alpha (α), delta (δ), and epsilon (ε).

According to another aspect, the present invention relates to a method for detecting a Hepatitis C Virus infection and/or a disease associated therewith in cells and/or a cell lysate, the method comprising the following steps: a) providing a sample of the cells or the cell lysate; and b) determining the activity, in the sample, of one or more proteins selected from the group consisting of casein kinase I alpha (α), delta (δ), and epsilon (ε).

As used herein, the term a “pharmaceutical(ly) effective amount” of a compound, or more specifically an inhibitor, is an amount effective to achieve the desired physiological result, either in cells treated in vitro or in a subject treated in vivo. Specifically, a pharmaceutically effective amount is an amount sufficient to inhibit, for some period of time, one or more of the clinically defined pathological processes associated with the viral infection. The effective amount may vary depending on the specific inhibitor selected, and is also dependent on a variety of factors and conditions related to the subject to be treated and the severity of the infection. For example, if the inhibitor is to be administered in vivo, factors such as the age, weight and health of the patient as well as dose response curves and toxicity data obtained in pre-clinical animal work would be among those factors considered. If the inhibitor is to be contacted with the cells in vitro, one would also design a variety of pre-clinical in vitro studies to assess such parameters as uptake, half-life, dose, toxicity, etc. The determination of a pharmaceutically effective amount for a given agent is well within the ability of those skilled in the art.

A therapeutically effective amount or dosage of a compound, such as an imidazole according to general formula (I), refers to that amount of the compound that results in an at least partial inhibition of virus production in the patient, which may be measured in several ways, e.g., reduction in HCV-DNA or HCV-Antigen levels in the patient's serum, and/or improvement in alanine amino transferase levels and liver histology and consequently results in a desired clinical benefit such as reduced viral load, suppression of progression of liver disease, and induction of immunological clearance or seroconversion. Toxicity and therapeutic efficacy of such compounds can be determined by standard pharmaceutical, pharmacological, and toxicological procedures in cell cultures or experimental animals for determining the LD50 (the dose lethal to 50% of the population) and the ED50 (the dose therapeutically effective in 50% of the population). The dose ratio between toxic and therapeutic effect is the therapeutic index and can be expressed as the ratio between LD50 and ED50. The dosage of the compound lies preferably within a range of circulating concentrations that include the ED50 with little or no toxicity. Preferably, the dosage of the compound corresponds to an effective concentration in the range of 0.05-30 μM, more preferably in the range of 0.1-10 μM. The actual amount of the composition administered will be dependent on the subject being treated, on the subject's weight, the severity of the affliction, the manner of administration and the judgement of the prescribing physician.

The transcription of DNA and the translation of RNA can be inhibited by oligonucleotides or oligonucleotide derivatives. Thus, the present invention discloses oligonucleotides and derivatives of oligonucleotides which may be used in the above-mentioned methods. The oligonucleotide and/or its derivatives bind to the DNA and/or RNA encoding the human cellular proteins casein kinase I α, δ, and/or ε and suppress the transcription of DNA or translation of RNA.

Some methods of the present invention identify compounds useful for prophylaxis and/or treatment of infections and/or diseases induced by HCV by screening a test compound, or a library of test compounds, for its ability to inhibit the above-mentioned human cellular proteins casein kinase I α, δ, and/or ε. A variety of assay protocols and detection techniques are well known in the art and easily adapted for this purpose by a skilled practitioner. Such methods include, but are not limited to, high throughput assays (e.g., kinase assays), and in vitro and in vivo cellular and tissue assays.

The present invention incorporates by reference in their entirety techniques well known in the field of molecular biology. These techniques include, but are not limited to, techniques described in the following publications: Ausubel, F. M. et al. eds., “Short Protocols In Molecular Biology” 4^{th} Ed. 1999, John Wiley & Sons, NY (ISBN 0-471-32938-X); Old, R. W. & S. B. Primrose “Principles of Gene Manipulation: An Introduction To Genetic Engineering” 3^{rd} Ed. 1985, Blackwell Scientific Publications, Boston. Studies in Microbiology: V.2, 409 pp. (ISBN 0-632-01318-4); Mayer, R. J. & J. H. Walker eds. “Immunochemical Methods In Cell and Molecular Biology” 1987, Academic Press, London. 325 pp. (ISBN 0-12480-855-7); Winnacker, E. L. “From Genes To Clones: Introduction To Gene Technology” 1987 VCH Publishers, NY. (translated by Horst Ibelgaufts) 634 pp. (ISBN 0-89573-614-4).

Yet another aspect of the present invention is directed to pharmaceutical compositions useful for the prophylaxis and/or treatment of an individual afflicted with HCV comprising at least one agent capable of inhibiting at least partially the activity of the human cellular proteins casein kinase I α, δ, and/or ε.

Therapeutics, pharmaceutically active agents or inhibitors, respectively, may be administered to cells from an individual in vitro, or may involve in vivo administration to the individual. Routes of administration of pharmaceutical preparations to an individual may include inhalation, oral and parenteral, including dermal, intradermal, intragastral, intracutan, intravasal, intravenous, intramuscular, intraperitoneal, intranasal, intravaginal, intrabuccal, percutan, rectal, subcutaneous, sublingual, topical or transdermal application, but are not limited the these ways of administration. For instance, the preferred preparations are in administratable form which is suitable for oral application. These administratable forms, for example, include pills, tablets, film tablets, coated tablets, capsules, powders and deposits. Administration to an individual may be in a single dose or in repeated administrations, and may be in any of a variety of physiologically acceptable salt forms, and/or with an acceptable pharmaceutical carrier, binder, lubricant, excipient, diluent and/or adjuvant. Pharmaceutically acceptable salt forms and standard pharmaceutical formulation techniques are well known to persons skilled in the art.

Still another aspect of the present invention relates to pharmaceutical compositions comprising a further ingredient active against HCV, like alpha interferon (Intron A, Schering-Plough; Roferon A, Hoffmann-La Roche; Wellferon, Glaxo Wellcome; Infergen, Amgen), ribavirin (Rebetol, Schering-Plough), and pegylated interferons (Pegasys, Hoffmann-La Roche; PEG Intron, Schering-Plough).

It is readily apparent to those skilled in the art that other suitable modifications and adaptations of the compositions and methods of the invention described herein may be made without departing from the scope of the invention or the embodiments disclosed herein. Having now described the present invention in detail, the same will be more clearly understood by reference to the following examples, which are included for purposes of illustration only and are not intended to be limiting of the invention.

### EXAMPLES

Kinase Purification

Confluent 15 cm dishes of Huh-7 cells were lysed in 500 μl lysis buffer per dish containing 20 mM Tris (tris-(hydroxymethyl)-amino methane) pH 8.0, 10 mM NaF (sodium fluoride), 2 mM MgCl₂ (Magnesium chloride), 1 mM DTT (dithiothreitol), 2 mM EGTA (ethylene glycol-bis(β-amino-ethyl ether)-N,N,N′,N′-tetraacetic acid), 1 mM PMSF (phenylmethylsulfonyl fluoride), 0.1% NP40 (Nonidet P-40), 10 μg/ml Aprotinin, 10 μg/ml Leupeptin, and 1 mM orthovanadate. Lysates were cleared by centrifugation (20 min, 13000 rpm, 4° C.) and filtered using a 0.45 μm syringe filter (Nalgene). This cleared material was further purified by a 1 ml Mono Q column (Amersham Biosciences) equilibrated with lysis buffer without additives. After washing the column, bound proteins were eluted with lysis buffer containing 1 M NaCl in a step gradient modus. Eluted proteins were collected in 1 ml fractions, systematically pooled, dialysed against 50 mM Tris pH 7.5, 10 mM NaF, 1 mM DTT, 0.01% Triton X-100 and stored at −80° C. Chromatography runs were performed on a AKTA explorer 10 system (Amersham Biosciences). Samples were subsequently assayed by in-vitro kinase assays and western blot analysis.

In-Vitro Kinase Assay

GST-NS5A immobilized to Glutathione Sepharose 4B beads (Amersham Biosciences) was washed twice with 500 μl kinase buffer (50 mM Tris pH 7.5, 10 mM MgCl₂, 0.5 mM DTT). Kinase reactions were performed in 50 μl kinase buffer supplemented with 10 μM ATP and 2 μCi [γ-³²P]ATP and a sample volume of 20 μl for 15 min at 37° C. under shaking. Reactions were stopped by addition of 7 μl 0.5 M EDTA (ethylene diamine tetraacetate) pH 8.0. After washing the beads, 30 μl 1.5×SDS sample buffer (50 mM Tris pH 6.8, 1.5% SDS, 15% glycerol, 2.5% β-mercaptothanol) were added. Samples were boiled for 5 minutes, subjected to gel electrophoresis on 11% gels and the Coomassie-stained gels were then autoradiographed.

For in vitro inhibitor testing compounds were preincubated with respective Mono Q fractions for 20 minutes and were also present during phosphorylation reaction. For linearity control in vitro kinase assays were carried out for 15 and 30 minutes respectively.

Immunoprecipitation of Mono Q Fractions and Immunoblotting

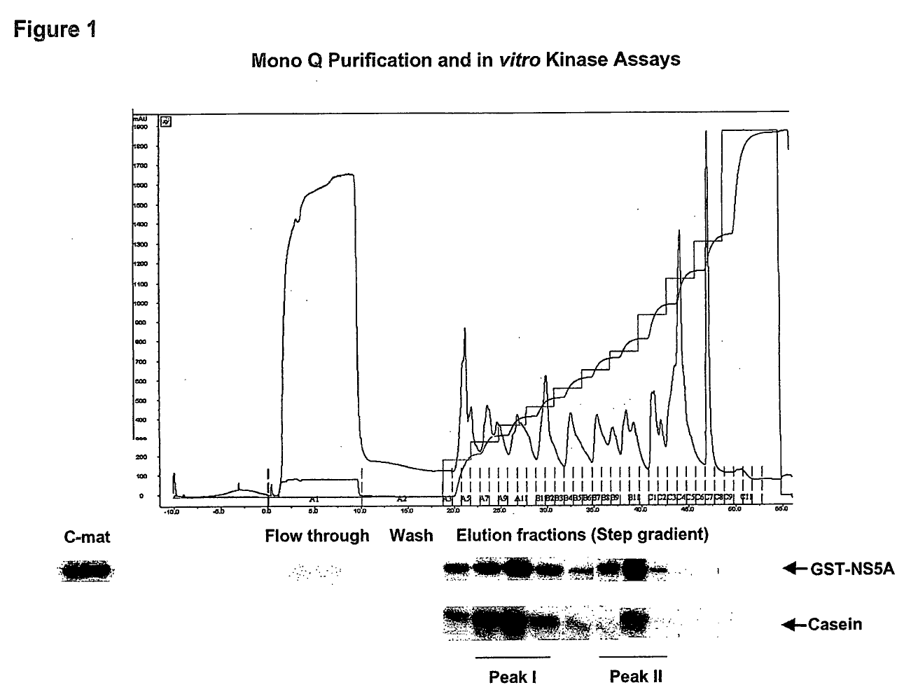

10 μg of αCKIα, αCKIδ, αCKIε antibodies (Santa Cruz) were bound to 20 μl equilibrated protein G Sepharose (Amersham Bioscience) in 300 μl 50 mM Tris pH 7.5, 150 mM NaCl, 0.5% Triton X-100 and 10% Glycerin for 2 h at 4° C. The protein G Sepharose-antibody conjugates were then washed twice with 50 mM Tris pH 7.5, 10 mM NaF, 1 mM DTT, 0.01% Triton X-100 and incubated with 160 μl of dialysed Mono Q fractions A10-A12 (see FIG. 1) for 2 h at 4° C. Immunodepleted supernatants were subsequently used for immunoblotting and in vitro kinase assays. Immunocomplexes bound to protein G Sepharose were washed twice with kinase buffer, eluted with SDS and fractionated by SDS-PAGE for immunobloting.

Proteins from SDS-gel were transferred onto nitrocellulose membrane (Schleicher & Schüll) by semi dry electro blotting. Prior to detection, the membrane was blocked in 12 mM Tris pH 7.5, 160 mM NaCl, 5% skim milk and 0.1% Triton X-100. Detection was carried out using primary antibodies: goat αCKIα, αCKIδ, αCKIε (Santa Cruz) and rabbit αCKII (Upstate Biotechnology) diluted 1:100 in the same buffer. Proteins were visualized using secondary antibody conjugated to HRP and a chemiluminescence detection system (Amersham Biosciences).

In-Gel Kinase Assay

Mono Q fractions were heated for 10 min at 50° C. in 3×SDS sample buffer. Sodium dodecylsulfate polyacrylamide gelelectrophoresis (SDS-PAGE) was performed on 11% minigels containing either 115 μg/ml GST or GST-NS5A protein co-polymerised in the separating gel. After electrophoresis, the gels were incubated twice for 30 min in 100 ml 20% isopropanol/50 mM Tris-HCl pH 8.0 and then washed for 1 hour in 250 ml 50 mM Tris-HCl pH 8.0/5 mM β-mercaptoethanol. To denature proteins, gels were incubated twice for 30 min in 100 ml 6 M guanidine-hydrochloride and then renatured for 16 hours in 250 ml 50 mM Tris-HCl pH8.0/5 mM β-mercaptoethanol/0.04% Tween 40 (polyoxyethylenesorbitan monopalmitate) at 4° C. (five changes). Gels were then equilibrated for 1 hour in 20 ml 40 mM HEPES-NaOH(N-[2-Hydroxyethyl]piperazine-N′-[2-ethansulfonicacid]) pH 8.0/2 mM DTT/10 mM MgCl₂. The kinase reaction was carried out for 1 hour in 15 ml 40 mM HEPES-NaOH pH 8.0/10 mM MgCl₂/0.5 mM EGTA/75 μCi [γ-³²P]ATP /10 μM ATP. Gels were then washed extensively in 5% TCA (trichloracetic acid)/1% sodium pyrophosphate until washes were free of radioactivity (usually five changes). Gels were then Coomassie-stained and dried and autoradiography was performed.

Cell Culture

Huh-7 were grown in Dulbecco's modified minimal essential medium (DMEM, Life Technologies GmbH, Karlsruhe, Germany) supplemented with 10% FCS (Fetal calf serum), 2 mM Glutamine, 1 mM Sodium Pyruvate, 100 U/ml penicillin and 100 μg/ml streptomycin at 7% CO₂.

Huh-5-15 cells were grown in DMEM supplemented with 10% FCS, 2 mM Glutamine, Penicillin (100 IU/ml)/Streptomycin (100 μg/ml and 1× nonessential amino acids in the presence of 1 mg/ml G418. Huh-5-2 cells were cultivated in the presence of 0.5 mg/ml G418. Cells were routinely passaged three times a week at a dilution of 1:3 or 1:2.

Plasmid Construction, Expression and Purification of Recombinant NS5A

All molecular techniques were performed according to standard procedures (Sambrook, J., E. F. Fritsch, and T. Maiatis, 1989, Molecular cloning: A Laboratory Manual, Cold Spring Harbor Laboratory Press, Cold Spring Harbor, N.Y.)

Plasmid pGEX-HCV-NS5A containing the entire coding sequence of HCV non-structural protein 5A (amino acid 1973 to 2490; Hepatitis C virus type 1b) was cloned in frame with the glutathione-S-transferase gene into the pGEX-5× (Pharmacia) vector.

E. coli XL1-Blue strain transformed with the pGEX-HCV-NS5A plasmid was cultured at 37° C. until the cell density reached a mid-log phase (A₆₀₀=0.6), followed by 1 mM isopropyl-1-thio-β-D-galactopyranoside induction for 4 hours. Bacterial pellets were resuspended in 50 mM Tris/HCl pH 7.5, 100 mM NaCl, 5 mM EDTA, 1% Trition X-100, 1 mM DTT, 10 μg/ml Aprotinin, 10 μg/ml Leupeptin, 0.5 mM PMSF and lysed by mild sonication. After centrifugation the supernatant was incubated with equilibrated Glutathione Sepharose 4B beads (Amersham Biosciences) over night at 4° C. Beads were washed and stored at 4° C. until use.

RNA Isolation and Metabolic Labeling

1×10⁶ Huh7-5-15 replicon cells and Huh-pcDNA3 cells were seeded into 10 cm culture plates and grown as described. After 24 hours cells were washed with phosphate-free medium and incubated in phosphate-free medium supplemented with 10% phosphate-free calf serum. Actinomycin D at a final concentration of 5 μg/ml was added to the cells together with the tested compounds at final concentrations of 10 and 50 μM. After 30 minutes of incubation cells were supplemented with 200 μCi ³³P-orthophosphate per dish and incubated over night. After cell lysis, RNA isolation was performed using the RNeasy mini kit (QIAGEN) according to recommendations given by the manufacturer. Northern blot was carried out according to standard procedures and autoradiography of the membrane was performed.

Compound Testing in the Huh-5-2 Replicon Cell Line

The Huh-5-2 cell line carries the persistant bicistronic replicon I389luc-ubi-neo/NS3-3′/5.1 that expresses a firefly luciferase-ubiquitin-neomycin phosphotransferase fusion protein under the control of the HCV 5′ UTR and the NS3-5B HCV polyprotein that harbors the cell culture adaptive mutation of NK5.1. (J. Virol. 75:4614-4624, 2001) and is driven by the EMCV-IRES. Autonomously replicating HCV RNA replicons are detectable by Northern blot or via expression of the luciferase part of the fusion protein in a luciferase reporter assay. For luciferase reporter assays, Huh-5-2 cells were seeded at 2500 cells per well in 96 well plates in medium without G418. After overnight incubation, compounds dissolved in DMSO were added to the medium at 20, 10, 5, 2.5 μM concentrations in duplicate samples. On day 3 after addition of compounds, Alamar Blue™ solution (Serotec), which contains a redox indicator, was added to the cells to measure cell proliferation and compound cytotoxicity. After incubation for 3-4 hours, fluorescence was monitored at the wavelengths of 560 nm and 590 nm with a Wallac 1420 multilabel counter. For the luciferase assay, the cells were subsequently washed twice with PBS without sodium hydrogen carbonate (Life Technologies GmbH, Karlsruhe), and luciferase activity was determined with the LucLite Plus Assay Kit (Packard Bioscience B.V.) according to the manufacturer's instructions in a Wallac 1450 Microbeta Luminescence counter.

Results

To identify the cellular kinase(s) responsible for NS5A phosphorylation, anion exchange chromatography using a Mono Q column was performed to fractionate the NS5A kinase activities from cytosolic extracts of Huh-7 cells. The bound proteins were eluted with a step gradient of NaCl as indicated in FIG. 1. Eluted protein fractions were assayed for in vitro phosphorylation of recombinant GST-NS5A fusion protein. The in vitro kinase assays performed revealed two separable peaks with fraction A10-A12 of peak I and fraction B10-B12 of peak II containing the highest specific activity (FIG. 1). Strikingly, a similar profile of eluted kinase activity could be observed when casein instead of GST-NS5A was used as a substrate (FIG. 1).

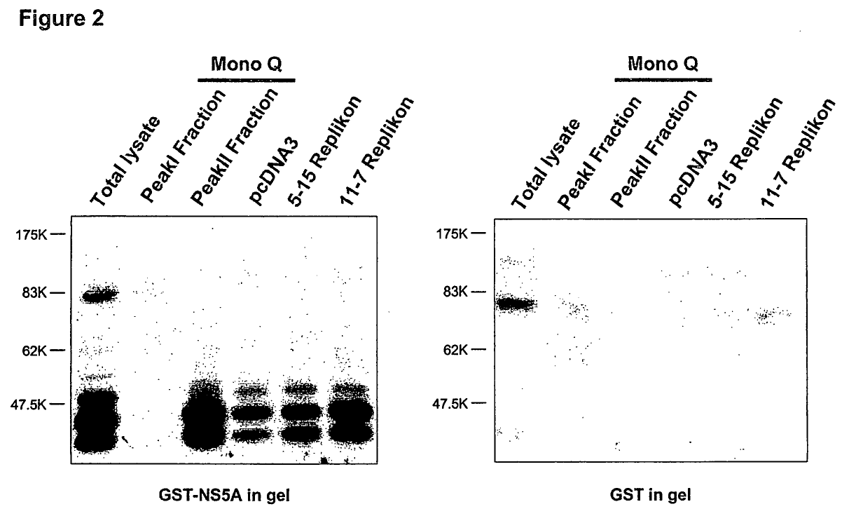

To further characterize the enzymes present in the peak I and peak II fractions, both fractions were subjected as well as total cell extracts from both parental and HCV replicon-carrying Huh-7 cells to SDS polyacrylamide gels containing either copolymerised GST or GST-NS5A and performed an in gel kinase assay upon renaturation of the proteins within the gels. In the GST-NS5A-containing gel two protein kinases of approximately 40 and 45 kDa were detected in the peak II fraction and the total cell extracts, but not in the gel polymerised with GST alone. This result indicated that GST-NS5A protein served as a substrate for the 40 and 45 kDa protein kinases. Moreover, no renaturable kinase activity was observed in the peak I fraction (FIG. 2), indicating that the NS5A kinases present in these fractions apparently do not refold properly to allow in gel detection and therefore exhibit biochemical properties distinct from NS5A kinases in the peak I fraction. These results indicate the existence of at least two different kinase activities in Huh7 cell extracts involved in the phosphorylation of NS5A with distinct renaturing properties.

Recent data indicate that the protein kinase CKII can catalyse NS5A phosphorylation in vitro. Interestingly, the apparent molecular weights of the peak II kinases detected in the in-gel kinase assay are comparable to those of the two catalytic subunits (α and α′) of CKII. Taken together, these data strongly suggest that kinase activity present in the peak II fractions can attributed to CKII. The observation, that the eluted protein fractions showed a similar pattern of phosphorylation using casein as kinase substrate (FIG. 1), suggested that CKI might be responsible for phosphorylation of NS5A by the protein kinases present in the peak I fractions.

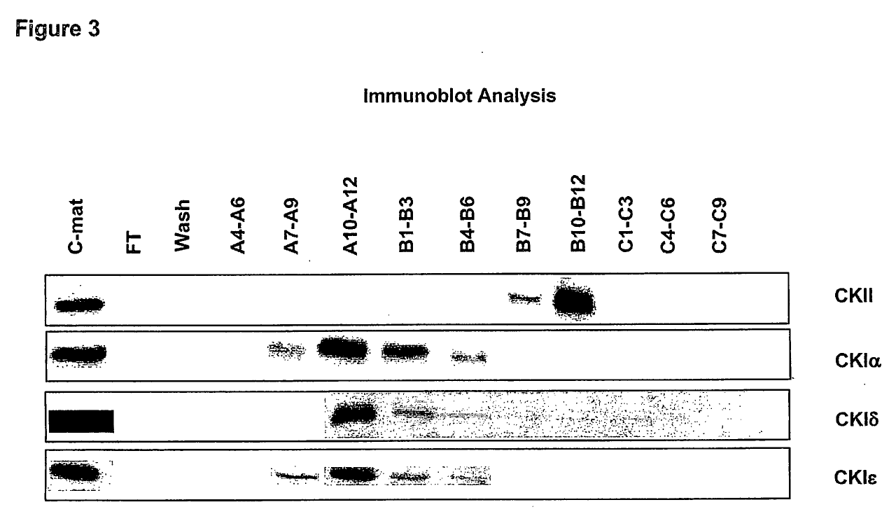

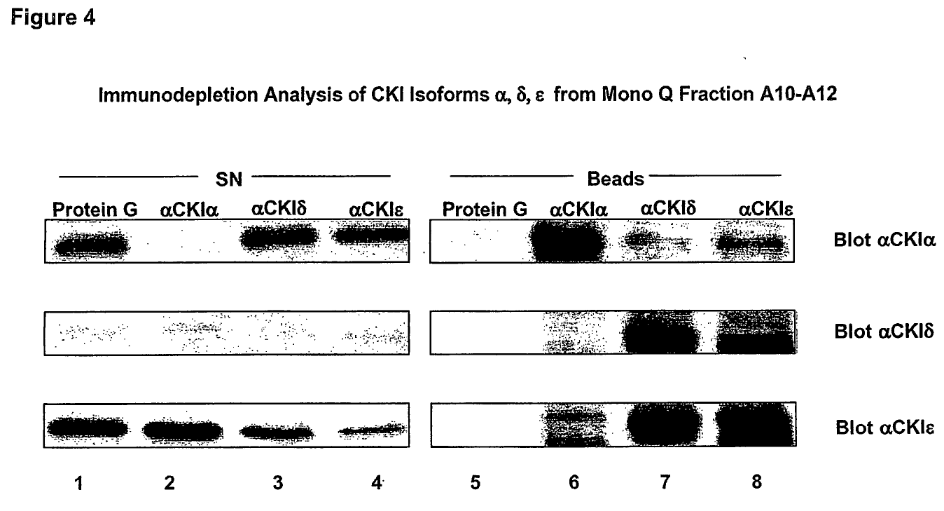

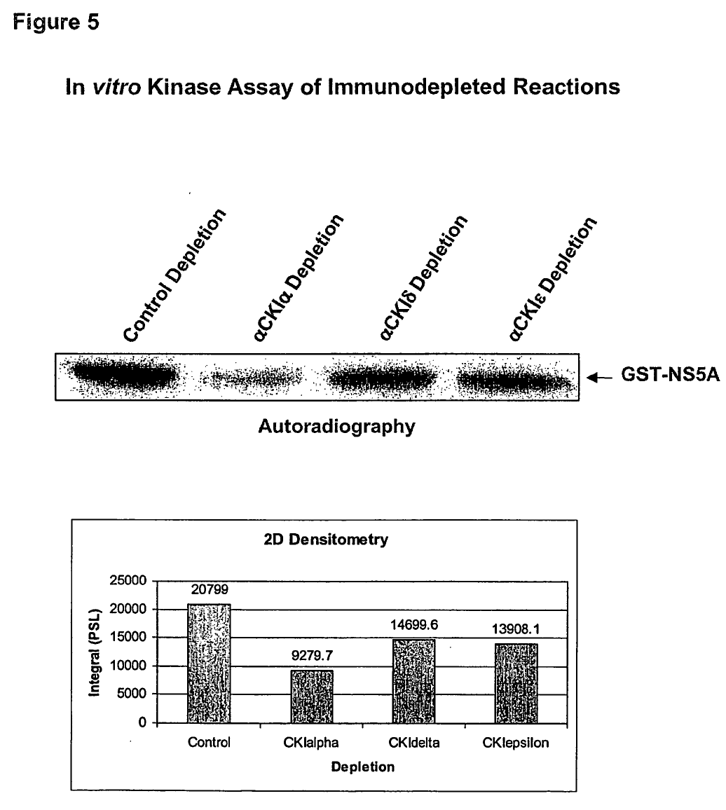

To test these assumptions, a Western blot analysis was performed on the eluted protein fractions from the Mono Q column using antibodies directed against CKII, CKIα, CKIδ and CKIε. As shown in FIG. 3, CKII was present only in the peak II fractions B7-B9 and B10-B12, while the α, δ and ε isoforms of CKI were detected in the peak I fractions A7-A9, A10-A12, B1-B3 and B4-B6. Interestingly, the highest concentrations of these kinases correlated with the maximum specific activity observed in a GST-NS5A-based in vitro kinase assay. The involvement of the CKI isoforms α, δand ε in the phosphorylation of NS5A was further confirmed by immunodepletion of these kinases from the Mono Q fraction A10-A12. For this purpose, dialysed aliquots of fraction A10-A12 were subjected to one round of immunodepletion in the absence of antibody (Protein G) or in the presence of anti-CKIα, anti-CKIδ or anti-CKIε antibody, respectively. Subsequently, Western blot analysis of the immunodepleted A10-A12 fractions (FIG. 4, lanes 1-4) and the immunoprecipitated CKI isoforms (FIG. 4, lanes 5-8) was performed. The immunodepleted supernatants were subsequently used to phosphorylate recombinant NS5A and a reduction of the relative phosphorylation levels of NS5A ranging between 44.6%-70.6% was observed (FIG. 5). This result strongly suggested that the NS5A phosphorylation detected is due to the presence of CKI in this fraction.

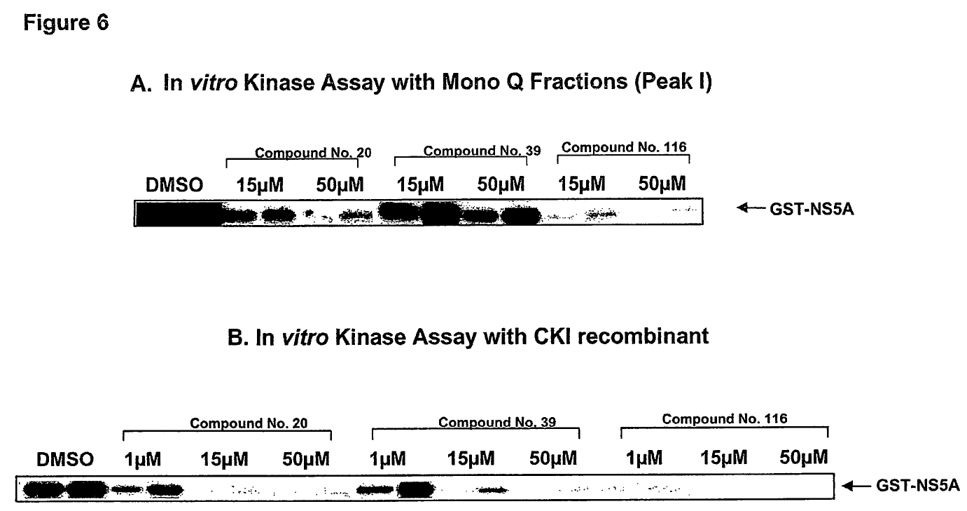

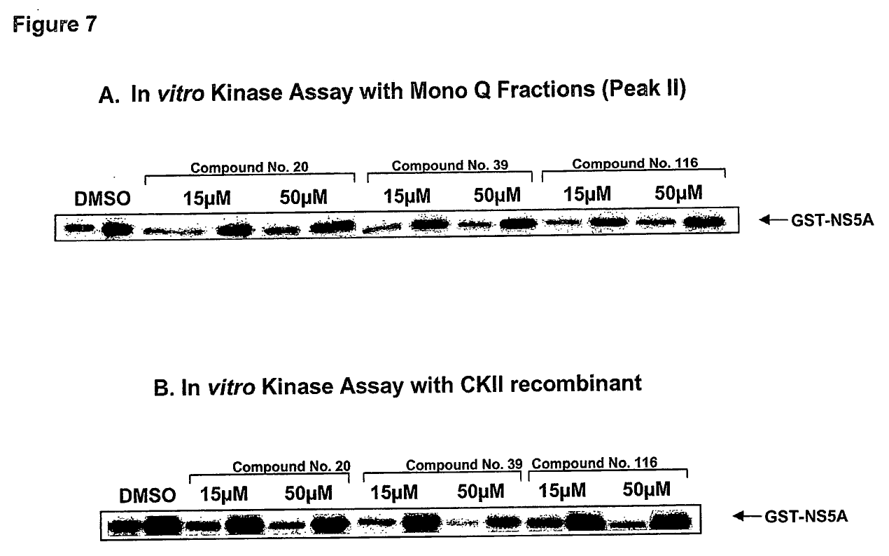

The above finding was further substantiated by including a panel of small molecule inhibitors in GST-NS5A phosphorylation reactions. In a first set of kinase assay using recombinant CKI and CKII, it was found that the three compounds listed in Table 1 can abolish CKI activity in vitro (FIG. 6B) without affecting CKII phosphorylation of NS5A (FIG. 7B). Subsequently, the role of the CKI isoforms α, δ and ε as NS5A phosphorylating kinases was confirmed by the strong inhibitory effects of these compounds on the kinase activity present in the Peak I fractions (FIG. 6A). Importantly, the inhibitory patterns of these compounds on NS5A phosphorylation by cellular kinases were comparable to that observed with recombinant CKI. In contrast, phosphorylation of NS5A was not significantly affected by pre-treatment of the peak II fractions with the same compounds (FIG. 7A).

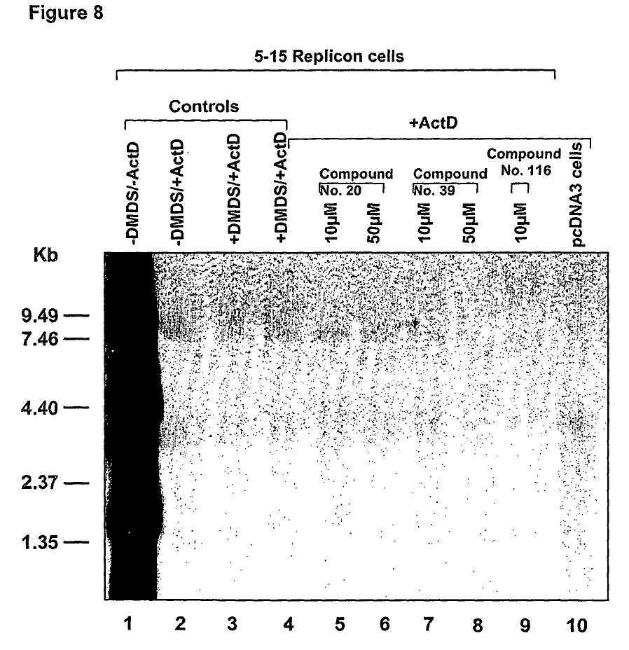

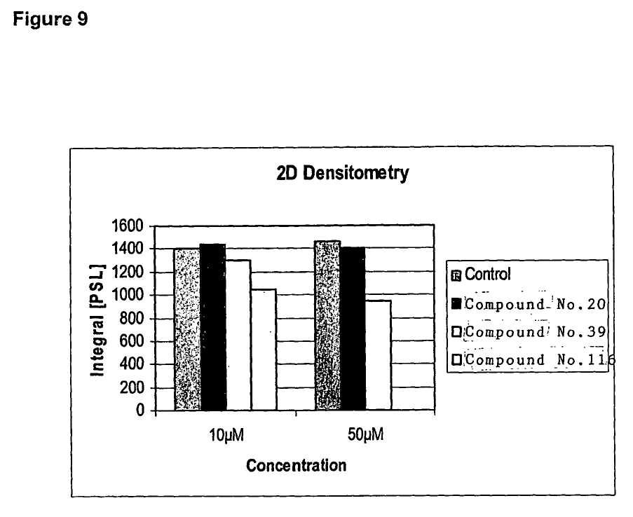

To test whether CKI inhibition has an impact on HCV replication, the HCV replicon cell line 5-15 was treated with the three compounds 4-[4-(4-fluoro-phenyl)-5-pyridine-4-yl-1H-imidazole-2-yl]-phenol (compound No. 20), 4-[5-(3-iodo-phenyl)-2-(4-methanesulfinyl-phenyl)-3H-imidazole-4-yl]-pyridine (compound No. 39), and 4-[4-(4-fluoro-phenyl)-5-pyridine-4-yl-1H-imidazole-2-yl]-benzylamine (compound No. 116) in the presence of actinomycin D, an antibiotic that selectively inhibits RNA synthesis from DNA but not RNA templates. Following overnight metabolic labelling with [³³P]-orthophosphate, newly synthesized radiolabeled RNA was isolated, blotted and visualized by autoradiography. As expected, the replication of HCV RNA of the expected molecular size of 9 kb was not affected by actinomycin D, whereas synthesis of cellular RNA was blocked (FIG. 8, lanes 2-9). In the presence of 50 μM 4-[5-(3-iodo-phenyl)-2-(4-methanesulfinyl-phenyl)-3H-imidazole-4-yl]-pyridine (compound No. 39), synthesis of HCV RNA was reduced and this was quantified on a phosphorimager using the Aida Image Analyzer software package (FIG. 9).

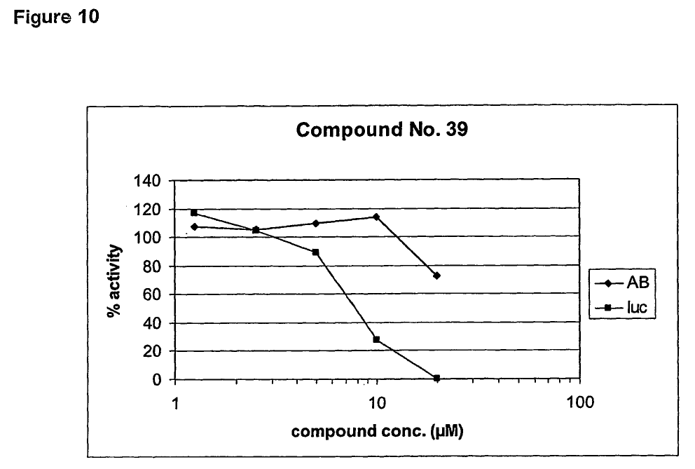

The inhibitory effect of the compound 4-[5-(3-iodo-phenyl)-2-(4-methanesulfinyl-phenyl)-3H-imidazole-4-yl]-pyridine (compound No. 39) on levels of HCV replicons was additionally tested in the Huh-5-2 replicon cell line. Huh-5-2 replicon cells, which carry subgenomic replicons expressing firefly luciferase in addition to the HCV replication-proteins were incubated with various concentrations of 4-[5-(3-iodo-phenyl)-2-(4-methanesulfinyl-phenyl)-3H-imidazole-4-yl]-pyridine for 3 days. Cellular toxicity of the compound was determined and luciferase activity measured as a function of replicon RNA levels. After normalization against background controls, mean values from the duplicate samples were expressed as percentage of the DMSO controls for the AlamarBlue (AB) assays as listed in Table 2 and luciferase reporter assays (luc) as listed in Table 3. The results are shown graphically in FIG. 10.

TABLE 1
Compounds

| Compound | name | structure |
|---|---|---|
| No. 20 | 4-[4-(4-fluoro-phenyl)- 5-pyridine-4-yl-1H- imidazole-2-yl]-phenol | 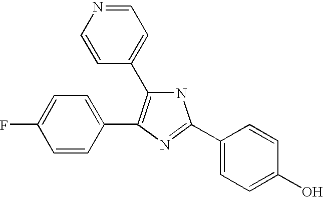 |
| No. 39 | 4-[5-(3-iodo-phenyl)- 2-(4-methanesulfinyl- phenyl)-3H-imidazole- 4-yl]-pyridine | 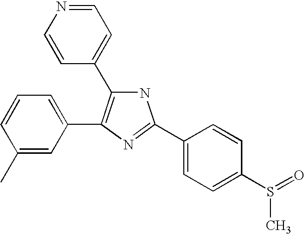 |
| No. 116 | 4-[4-(4-fluoro-phenyl)- 5-pyridine-4-yl-1H- imidazole-2-yl]- benzylamine | 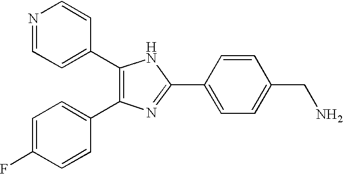 |

TABLE 2
Effect of 4-[5-(3-iodo-phenyl)-2-(4-methanesulfinyl-phenyl)-3H-
imidazole-4-yl]-pyridine (compound No. 39) on viability of Huh-5-2
replicon cells by the Alamar Blue toxicity assay.

| | Compound No. |
|---|---|
| | 39% |
| | of cell |
| conc. (μM) | viability |
| 20 | 72 |
| 10 | 114 |
| 5 | 109 |
| 2.5 | 105 |
| 1.25 | 108 |

TABLE 3
Effect of 4-[5-(3-iodo-phenyl)-2-(4-methanesulfinyl-phenyl)-3H-
imidazole-4-yl]-pyridine (compound No. 39) on autonomous replication
of HCV replicons in the Huh-5-2 cell line by luciferase reporter assay.

| | Compound No. |
|---|---|
| conc. (μM) | 39% activity |
| 20 | 0 |
| 10 | 28 |
| 5 | 89 |
| 2.5 | 104 |
| 1.25 | 117 |

Toxicity was low for 4-[5-(3-iodo-phenyl)-2-(4-methanesulfinyl-phenyl)-3H-imidazole-4-yl]-pyridine (compound No. 39) up to a concentration of 10 μM. Replication of subgenomic HCV replicon RNA as measured in the luciferase reporter assay was reduced for compound No. 39 with an IC50 of 9 μM.

From the results observed with 4-[5-(3-iodo-phenyl)-2-(4-methanesulfinyl-phenyl)-3H-imidazole-4-yl]-pyridine, which is a potent inhibitor of the NS5A-phosphorylating activity in the CKI-enriched partially purified fraction, inhibitors of CKI α, δ or ε are indicated to be potent inhibitors of Hepatitis C viral replication.

By biochemical purification of Huh7 total cell extracts of two NS5A phosphorylating activity peaks with distinct biochemical properties were detected. By further biochemical characterization, the following human proteins were identified as NS5A phosphorylating kinases and therefore potential anti-HCV targets: CKI α, δ and ε. Small molecular weight compounds from the class of imidazoles were revealed as inhibitors of CKI and as candidates for anti-HCV treatment.

1. A compound having the general formula (I): 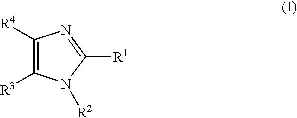
wherein:
R¹, R^{1′}, and R^{1″} represent independently of each other 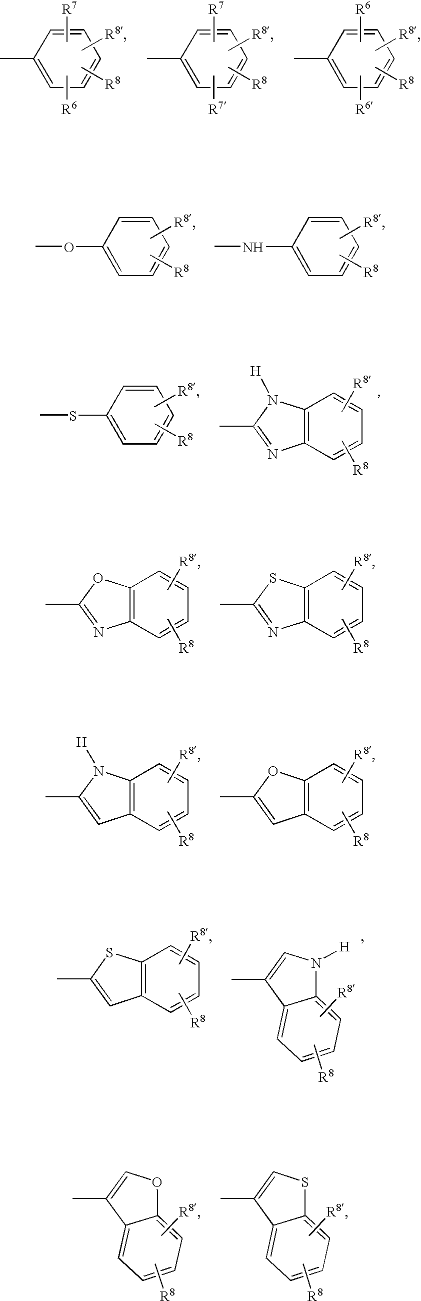 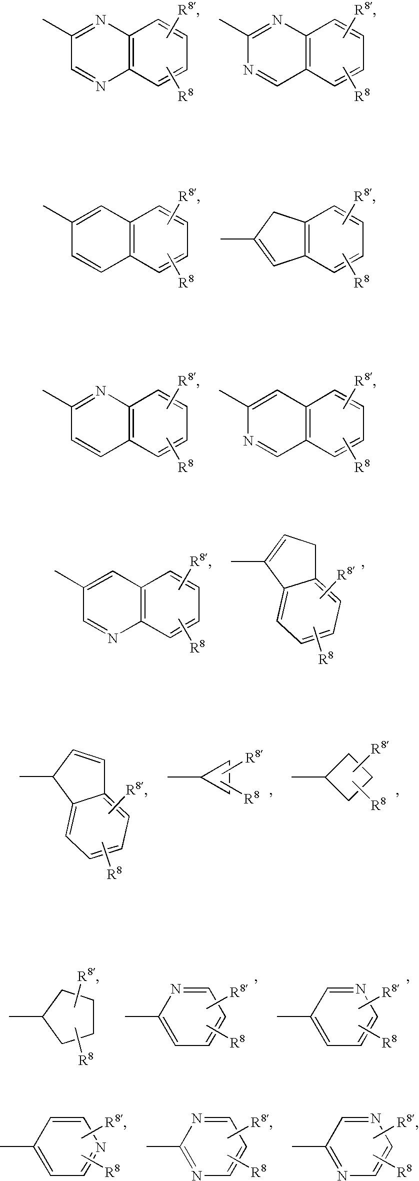 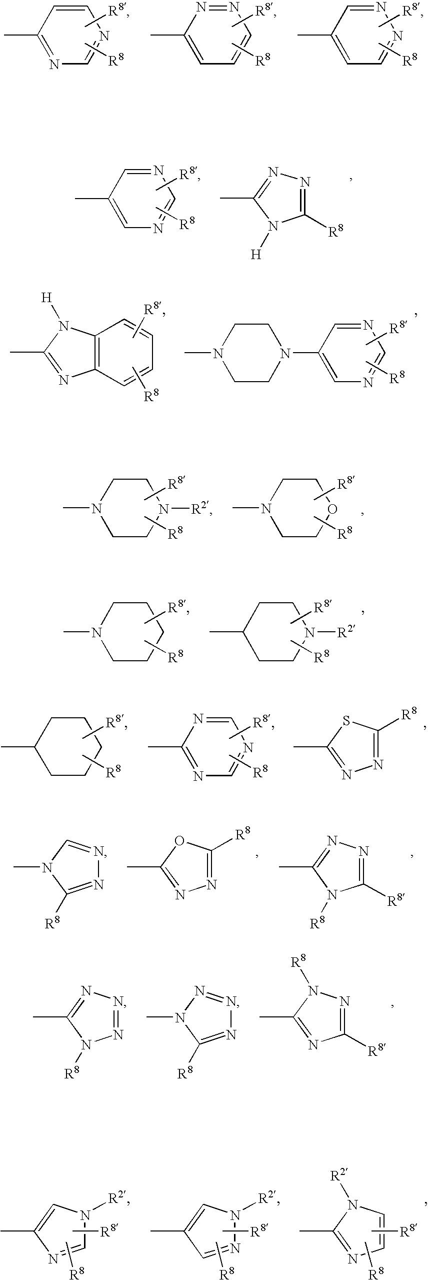 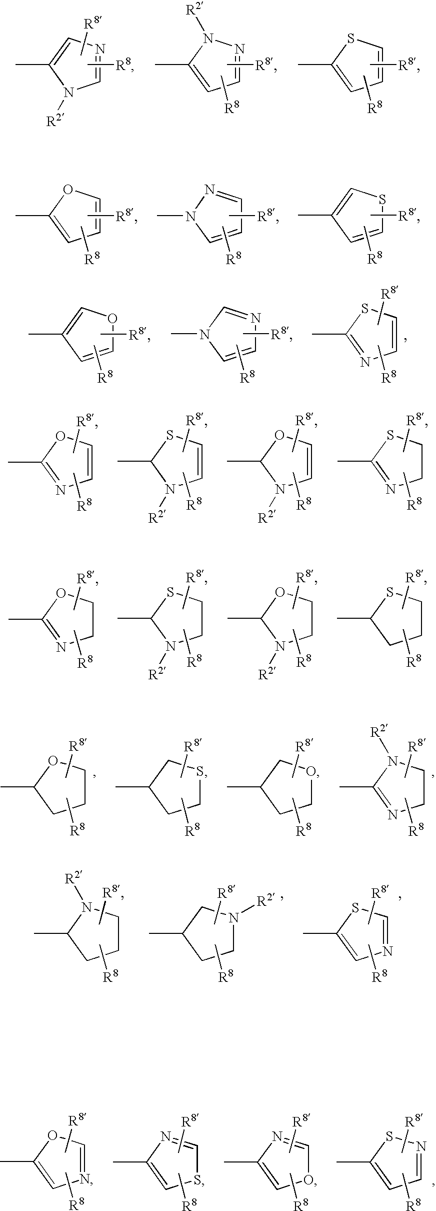 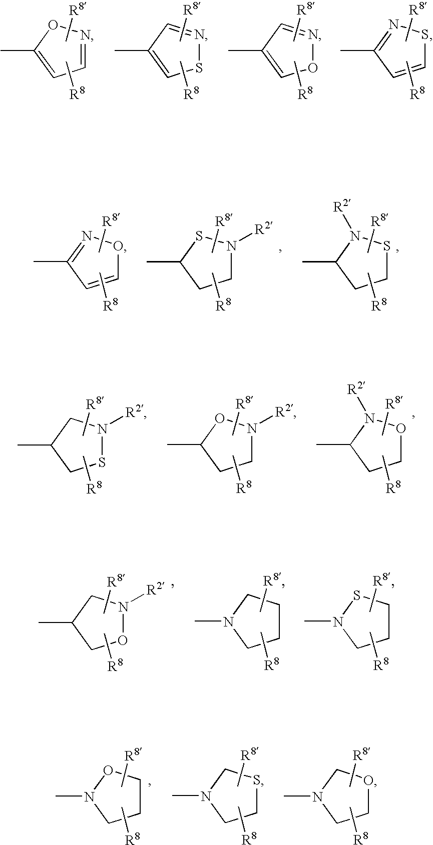
R², R^{2′}, and R^{2″} represent independently of each other —H, —CH₃, —C₂H₅, —CH═CH₂, —C≡CH, —C₃H₇, -cyclo-C₃H₅, —CH(CH₃)₂, —CH₂—CH═CH₂, —C(CH₃)═CH₂, —CH═CH—CH₃, —C≡C—CH₃, —CH₂—C≡CH, —C₄H₉, -cyclo-C₄H₇, —CH₂—CH(CH₃)₂, —CH(CH₃)—C₂H₅, —C(CH₃)₃, —C₅H₁₁, -cyclo-C₅H₉, —C₆H₁₃, -cyclo-C₆H₁₁, -Ph, —C(R⁵)₃, —C(R^{5′})₃, —CR⁵(R^{5′})₂, —CR⁵(R^{5′})R^{5″}, C₂(R⁵)₅, —CH₂C(R⁵)₃, CH₂—C(R^{5′})₃, —CH₂—CR⁵(R^{5′})₂, —CH₂—CR⁵(R^{5′})R^{5″}, C₃(R⁵)₇, —C₂H₄—C(R⁵)₃, —C₇H₁₅, -cyclo-C₇H₁₃, —CH₂Ph, —C₈H₁₇, -cyclo-C₈H₁₅, —C₂H₄Ph, —CH═CH-Ph, —C≡C-Ph;
R³ and R⁴ represent independently of each other —R^{1′}, —R^{1″}, —R⁶, —R^{6—}, —OR^{2′}, —OR^{2″}, —SR^{2′}, —SR^{2″};
R⁵, R^{5′} and R^{5″} represent independently of each other —F, —Cl, —Br, —I, —CN;
R⁶ and R^{6′} represent independently of each other —R^{2′}, —R^{2″}, —C₂H₄—H═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C≡C—CH₃, —C≡C—CH═CH₂, —CH═CH—C≡CH, —C≡C—C≡CH, —C₂H₄—CH(CH₃)₂, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₃H₆—CH═CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, —C(CH₃)═C(CH₃)₂, —C₃H₆—C≡CH, —C≡C—C₃H₇, —C₂H₄—C≡C—CH₃, —CH₂C≡C—C₂H₅, —CH₂—C≡C—CH═CH₂, —CH₂—CH═CH—C≡CH, —CH₂—C≡C—C≡CH, —C≡C—CH═CH—CH₃, —CH═CH—C≡C—CH₃, —C≡C—C≡C—CH₃, —C≡C—CH₂—CH═CH₂, —CH═CH—CH₂—C≡CH, —C≡C—CH₂—C≡CH, —C(CH₃)═CH—CH═CH₂, —CH═C(CH₃)—CH═CH₂, —CH═CH—C(CH₃)═CH₂, —C(CH₃)═CH—C≡CH, —CH═C(CH₃)—C≡CH, —C≡C—C(CH₃)═CH₂, —C₃H₆—CH(CH₃)₂, —C₂H₄—CH(CH₃)—C₂H₅, —CH(CH₃)—C₄H₉, —CH₂CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH═CH₂, —CH═CH—C₄H₉, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C₄H₈—C≡CH, —C≡C—C₄H₉, —C₃H₆—C≡C—CH₃, —CH₂—C≡C—C₃H₇, —C₂H₄—C≡C—C₂H₅, -o-C₆H₄—R², -o-C₆H₄—R^{2′}, -m-C₆H₄—R², -m-C₆H₄—R^{2′}, -p-C₆H₄—R², -p-C₆H₄—R^{2′}, -o-CH₂—C₆H₄—R², -o-CH₂—C₆H₄—R^{2′}, -m-CH₂C₆H₄—R², -m-CH₂—C₆H₄—R^{2′}, -p-CH₂—C₆H₄—R², -p-CH₂—C₆H₄—R^{2′};
R⁷ and R^{7′} represent independently of each other —R^{5′}, —R^{5″}, —H, —NO₂, —NO, —N₃, —OCN, —NCO, —SCN, —NCS, —COCN, —COOR^{2′}, —COOR^{2″}, —CO—R^{2′}, —CO—R^{2″}, CONR^{2′}R^{2″}, —NR^{2′}R^{2″}, —NR⁶R^{6′}, —N^{⊕}R^{2′}, R^{2″}R^{6′}, —SOR^{2′}, —SOR^{2″}, —SO₂R^{2′}, —SO₂R^{2″}, —SO₃R^{2′}, —SO₃R^{2″}, —NHCO—R^{2′}, —NHCO—R^{2″}, —NHCOO—R^{2′}, —NHCOO—R^{2″}, —OCONR^{2′}R^{2″}, —OCONR⁶R^{6′}, —OCOR^{2′}, —OCOR^{2″}, —NH—SO₂—R^{2′}, —NH—SO₂—R^{2″}, —SO₂—NR^{2′}R^{2″}, —SO₂—NR⁶R^{6′}, —NH—CO—NH—R^{2′}, —NH—CO—NH—R^{2″}, —NH—CS—NH—R^{2′}, —NH—CS—NH—R^{2″}, —OR^{2′}, —OR^{2—}, —SR^{2′}, —SR^{2″};
R⁸ and R^{8′} represent independently of each other —R⁷, —R^{7′}, —R^{6′}, —R^{6″};
and pharmaceutically acceptable salts thereof

2. The compound according to claim 1, wherein
R¹, R^{1′}, and R^{1″} represent independently of each other 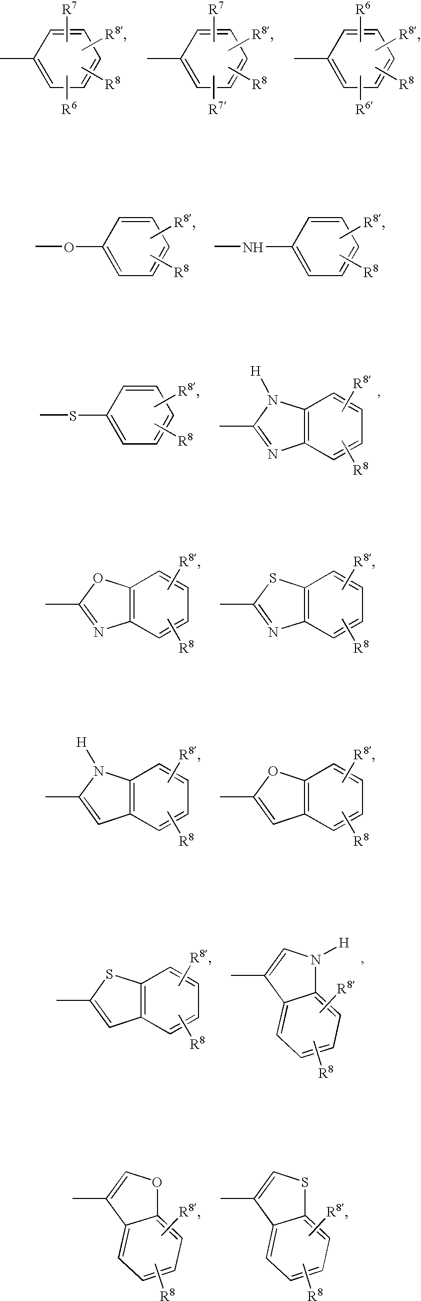 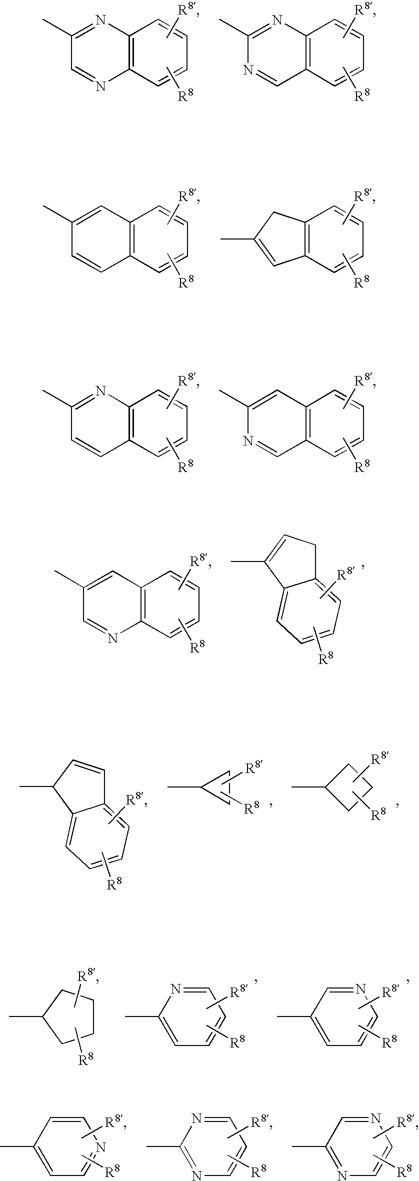 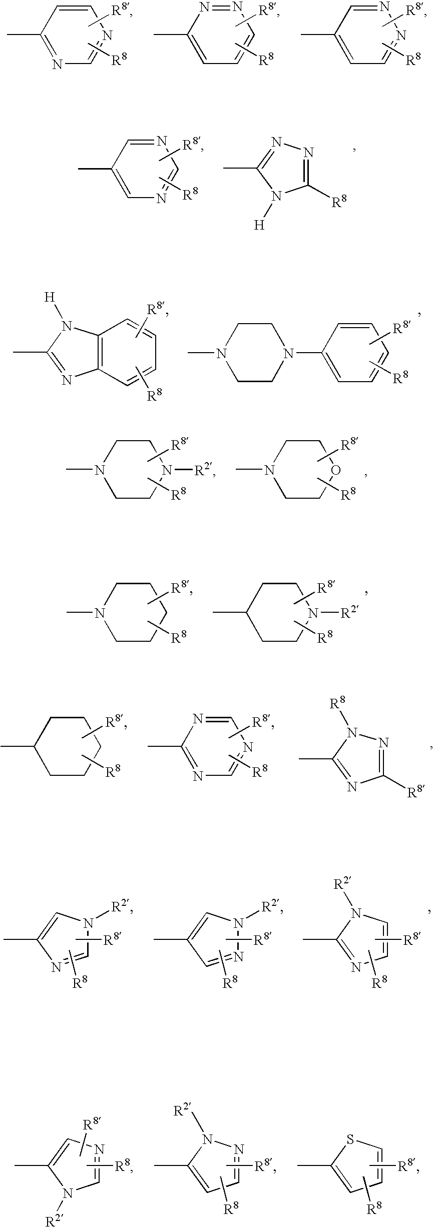 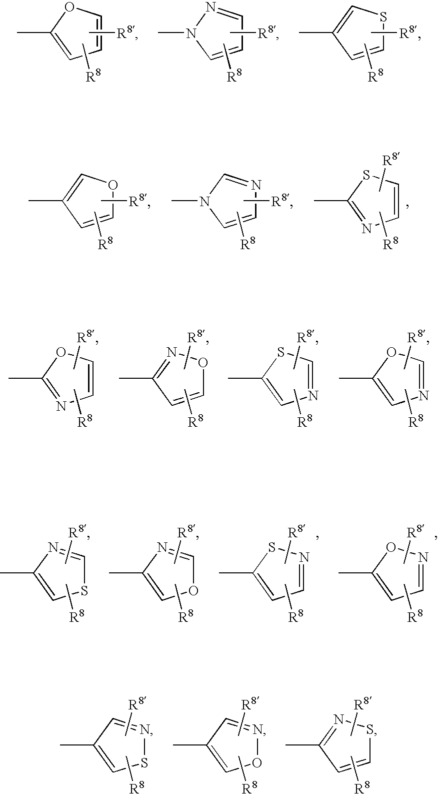
and R^{2′}, R⁶, R^{6′}, R⁷, R^{7′}, R⁸, R^{8′} have the meanings as defined in claim 1.

3. The compound according to claim 2, wherein
R¹, R^{1′}, and R^{1″} represent independently of each other 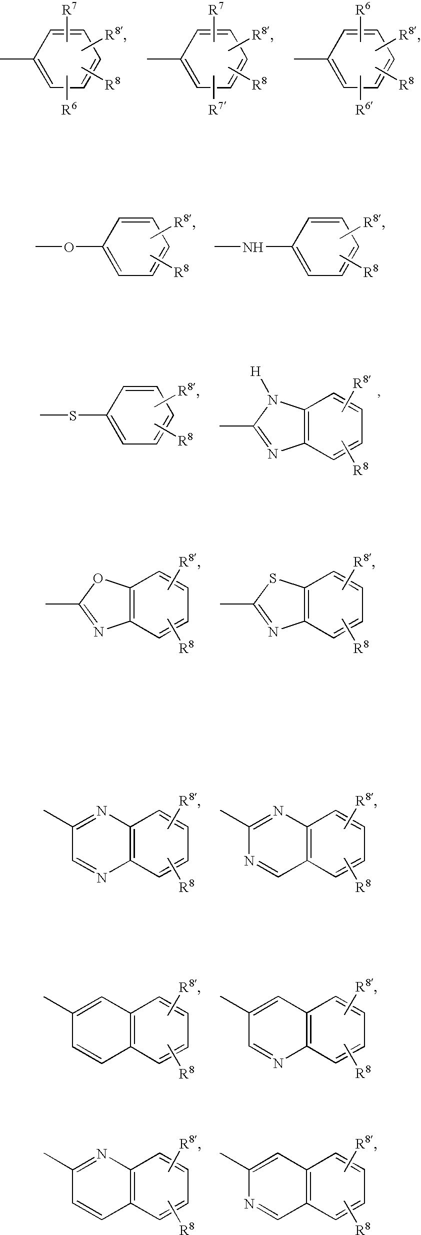 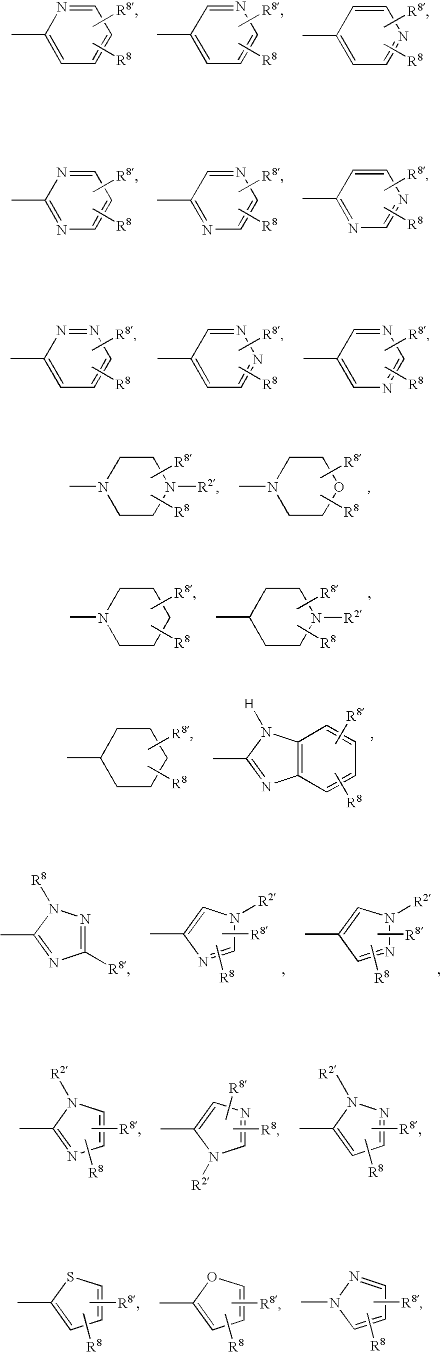 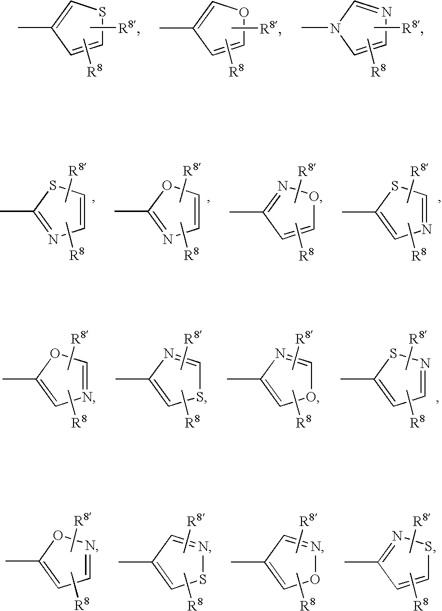
and R^{2′}, R⁶, R^{6′}, R⁷, R^{7′}, R⁸, R^{8′} have the meanings as defined in claim 1.

4. The compound according to claim 3, wherein
R¹, R^{1′}, and R^{1″} represent independently of each other 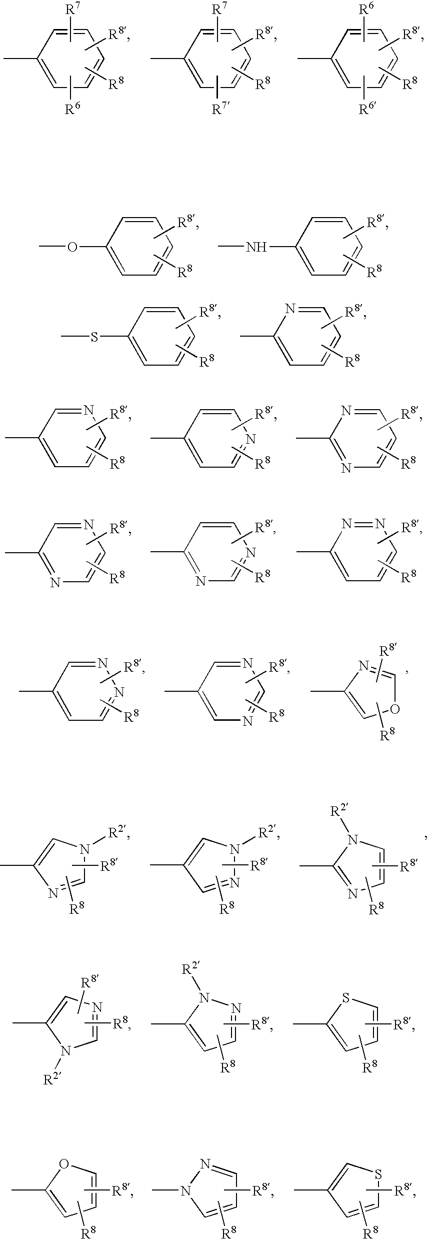 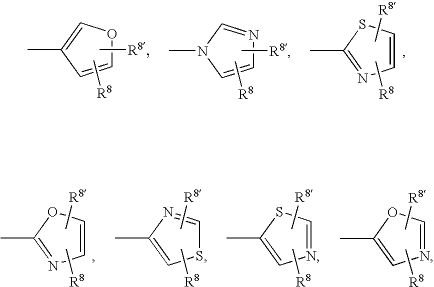
and R^{2′}, R⁶, R^{6′}, R⁷, R^{7′}, R⁸, R^{8′} have the meanings as defined in claim 1.

5. The compound according to claim 1, wherein
R², R^{2′}, and R^{2″} represent independently of each other —H, —CH₃, —C₂H₅, —CH═CH₂, —C≡CH, —C₃H₇, -cyclo-C₃H₅, —CH(CH₃)₂, —CH₂—CH═CH₂, —C₄H₉, -cyclo-C₄H₇, —CH₂—CH(CH₃)₂, —CH(CH₃)—C₂H₅, —C(CH₃)₃, —C₅H₁₁, -cyclo-C₅H₉, —C₆H₁₃, -cyclo-C₆H₁₁, -Ph, —C(R⁵)₃, —C(R^{5′})₃, —CR⁵(R^{5′})₂, —CH₂Ph; and R⁵, R^{5′} have the meanings as defined in claim 1.

6. The compound according to claim 1, wherein
R⁵, R^{5′} and R^{5″} represent independently of each other —F, —Cl, —Br.

7. The compound according to claim 1, wherein
R⁶ and R^{6′} represent independently of each other —R^{2′}, —R^{2″}, -o-C₆H₄—R², -o-C₆H₄—R^{2′}, -m-C₆H₄—R², -m-C₆H₄—R^{2′}, -p-C₆H₄—R², -p-C₆H₄—R^{2′}, -o-CH₂—C₆H₄—R², -o-CH₂—C₆H₄—R^{2′}, -m-CH₂—C₆H₄—R², -m-CH₂—C₆H₄—R^{2′}, -p-CH₂—C₆H₄—R², -p-CH₂—C₆H₄—R^{2′};
and R², R^{2′}, R^{2″} have the meanings as defined in claim 1.

8. The compound according to claim 7, wherein
R⁶ and R^{6′} represent independently of each other —H, —CH₃, —C₂H₅, —CH═CH₂, —C≡CH, —C₃H₇, -cyclo-C₃H₅, —CH(CH₃)₂, —CH₂—CH═CH₂, —C₄H₉, -cyclo-C₄H₇, —CH₂—CH(CH₃)₂, —CH(CH₃)—C₂H₅, —C(CH₃)₃, —C₅H₁₁, -cyclo-C₅H₉, —C₆H₁₃, -cyclo-C₆H₁₁, -Ph, —C(R⁵)₃, —C(R^{5′})₃, —CR⁵(R^{5′})₂, —CH₂Ph, -o-C₆H₄—CH₃, -o-C₆H₄—C₂H₅, -m-C₆H₄—CH₃, -m-C₆H₄—C₂H₅, -p-C₆H₄—CH₃, p-C₆H₄—C₂H₅, -o-CH₂—C₆H₄—CH₃, -o-CH₂C₆H₄—C₂H₅, -m-CH₂—C₆H₄—CH₃, -m-CH₂—C₆H₄C₂H₅, -p-CH₂—C₆H₄—CH₃, -p-CH₂—C₆H₄—C₂H₅;
and R⁵, R^{5′} have the meanings as defined in claim 1.

9. The compound according to claim 1, wherein
R⁷ and R^{7′} represent independently of each other —F, —Cl, —Br, —H, —NO₂, —COOR^{2′}, —COOR^{2″}, —CO—R^{2′}, —CO—R^{2″}, —CONR^{2′}R^{2″}, —NR^{2′}R^{2″}, —NR⁶R^{6′}, —SOR^{2′}, —SOR^{2″}, —SO₂R^{2′}, —SO₂R^{2″}, —SO₃R^{2′}, —SO₃R^{2″}, —NHCO—R^{2′}, —NHCO—R^{2″}, —OCOR^{2′}, —OCOR^{2″}, —OR^{2′}, —OR^{2″}, —SR^{2′}, —SR^{2″};
and R^{2′}, R^{2″}, R⁶, R^{6′} have the meanings as defined in claim 1.

10. The compound according to claim 1
(Compound 1) 4-[5-(4-Fluoro-phenyl)-2-(4-isopropyl-phenyl)-3H-imidazole-4-yl]-pyridine,
(Compound 2) 3-[4-(4-Fluoro-phenyl)-5-pyridine-4-yl-1H-imidazole-2-yl]-4-nitro-phenol,
(Compound 3) 4-[4-(4-Fluoro-phenyl)-5-pyridine-4-yl-1H-imidazole-2-yl]-2-nitro-phenol,
(Compound 4) 4-[5-(4-Fluoro-phenyl)-2-(3-trifluoromethyl-phenyl)-3H-imidazole-4-yl]-pyridine,
(Compound 5) 2,6-Di-tert-butyl-4-[4-(4-fluoro-phenyl)-5-pyridine-4-yl)-1H-imidazole-2-yl]-phenol,
(Compound 6) 4-[2-(2,5-Bis-trifluoromethyl)-5-(4-fluoro-phenyl)-3H-imidazole-4-yl]-pyridine,
(Compound 7) 4-[5-(4-Fluoro-phenyl)-2-furan-2-yl-3H-imidazole-4-yl]-pyridine,
(Compound 8) 4-[5-(4-Fluoro-phenyl)-2-(2-methoxy-phenyl)-3H-imidazole-4-yl]-pyridine,
(Compound 9) 4-[5-(4-Fluoro-phenyl)-2-(5-methyl-furan-2-yl)-3H-imidazole-4-yl]-pyridine,
(Compound 10) 4-[5-(4-Fluoro-phenyl)-2-(3-methoxy-phenyl)-3H-imidazole-4-yl]-pyridine,
(Compound 11) 4-[5-(4-Fluoro-phenyl)-2-p-tolyl-3H-imidazole-4-yl]-pyridine,
(Compound 12) 4-[5-(4-Fluoro-phenyl)-2-(4-methoxy-phenyl)-3H-imidazole-4-yl]-pyridine,
(Compound 13) 4-[5-(4-Fluoro-phenyl)-2-(2-chloro-phenyl)-3H-imidazole-4-yl]-pyridine,
(Compound 14) 4-[5-(4-Fluoro-phenyl)-2-(2,4,6-trimethyl-phenyl)-3H-imidazole-4-yl]-pyridine,
(Compound 15) 4-[5-(4-Fluoro-phenyl)-2-(2,4-dichloro-phenyl)-3H-imidazole-4-yl]-pyridine,
(Compound 16) 4-[5-(4-Fluoro-phenyl)-2-(2,3-dichloro-phenyl)-3H-imidazole-4-yl]-pyridine,
(Compound 17) 4-[4-(4-Fluoro-phenyl)-5-pyridine-4-yl)-1H-imidazole-2-yl]-2-methoxy-phenol,
(Compound 18) 4-[5-(4-Fluoro-phenyl)-2-(2-nitro-phenyl)-3H-imidazole-4-yl]-pyridine,
(Compound 19) 4-[4-(4-Fluoro-phenyl)-5-pyridine-4-yl-1H-imidazole-2-yl]-benzene-1,2-diol,
(Compound 20) 4-[4-(4-Fluoro-phenyl)-5-pyridine-4-yl-1H-imidazole-2-yl]-phenol,
(Compound 21) 4-[2-(4,5-Dimethoxy-2-nitro-phenyl)-5-(4-fluoro-phenyl)₇₃H-imidazole-4-yl]-pyridine,
(Compound 22) 4-[5-(4-Fluoro-phenyl)-2-(3-chloro-phenyl)-3H-imidazole-4-yl]-pyridine,
(Compound 23) 4-[5-(4-Fluoro-phenyl)-2-(3-bromo-phenyl)-3H-imidazole-4-yl]-pyridine,
(Compound 24) 4-[5-(4-Fluoro-phenyl)-2-(3-nitro-phenyl)-3H-imidazole-4-yl]-pyridine,
(Compound 25) 4-[5-(4-Fluoro-phenyl)-2-(4-nitro-phenyl)-3H-imidazole-4-yl]-pyridine,
(Compound 26) 4-[5-(4-Fluoro-phenyl)-2-naphtalene-1-yl-3H-imidazole-4-yl]-pyridine,
(Compound 27) 4-[2-(3,5-Bis-trifluoromethyl-phenyl)-5-(4-fluoro-phenyl)-3H-imidazole-4-yl]-pyridine,
(Compound 28) {4-[4-(4-Fluoro-phenyl)-5-pyridine-4-yl-1H-imidazole-2-yl]-phenyl}-dimethyl-amine
(Compound 29) 4-[5-(4-Fluoro-phenyl)-2-(3,4-dichloro-phenyl)-3H-imidazole-4-yl]-pyridine,
(Compound 30) 4-[5-(4-Fluoro-phenyl)-2-(4-trifluoromethyl-phenyl)-3H-imidazole-4-yl]-pyridine,
(Compound 31) 4-[4-(4-Fluoro-phenyl)-5-pyridine-4-yl-1H-imidazole-2-yl]-2,6-dimethyl-phenol,
(Compound 32) 4-[5-(4-Fluoro-phenyl)-2-(4-methylsulfanyl-phenyl)-3H-imidazole-4-yl]-pyridine,
(Compound 33) 3-[4-(4-Fluoro-phenyl)-5-pyridine-4-yl-1H-imidazole-2-yl]-1H-indole,
(Compound 34) 4-[5-(4-Fluoro-phenyl)-2-(4-chloro-phenyl)-3H-imidazole-4-yl]-pyridine,
(Compound 35) 4-[5-(4-Fluoro-phenyl)-2-thiophene-2-yl-3H-imidazole-4-yl]-pyridine
(Compound 36) 4-[5-(4-Fluoro-phenyl)-2-(4-bromo-phenyl)-3H-imidazole-4-yl]-pyridine,
(Compound 37) 4-[2-(3,4-Dimethoxy-phenyl)-5-(4-fluoro-phenyl)-3H-imidazole-4-yl]-pyridine,
(Compound 38) 4-[5-(4-Fluoro-phenyl)-2-(4-methanesulfinyl-phenyl)-3H-imidazole-4-yl]-pyridine,
(Compound 39) 4-[5-(3-Iodo-phenyl)-2-(4-methanesulfinyl-phenyl)-3H-imidazole-4-yl]-pyridine,
(Compound 40) 6-(4-Fluoro-phenyl)-5-pyridine-4-yl-3,7-dihydro-2H-imidazole-[2,1-b]thiazole,
(Compound 41) 4-[5-Ethyl-2-(4-methoxy-phenyl)-1H-imidazole-4-yl]-pyridine,
(Compound 42) 4-[2,5-Bis-(4-chloro-phenyl)-1H-imidazole-4-yl]-pyridine,
(Compound 43) 4-[2-(4-Bromo-phenyl)-5-(4-chloro-phenyl)-1H-imidazole-4-yl]-pyridine,
(Compound 44) 4-[2-(2-Chloro-phenyl)-5-(4-chloro-phenyl)-1H-imidazole-4-yl]-pyridine,
(Compound 45) 4-[2-(3-Bromo-phenyl)-5-(4-chloro-phenyl)-1H-imidazole-4-yl]-pyridine,
(Compound 46) 4-[5-(4-Chloro-phenyl)-2-(2,3-dichloro-phenyl)-1H-imidazole-4-yl]-pyridine,
(Compound 47) 3-[5-(4-Chloro-phenyl)-4-pyridine-4-yl-1H-imidazole-2-yl]-4-nitro-phenol,
(Compound 48) 4-[5-(4-Chloro-phenyl)-2-(4-fluoro-phenyl)-1H-imidazole-4-yl]-pyridine,
(Compound 49) 4-[5-(4-Chloro-phenyl)-2-naphtalene-1-yl-1H-imidazole-4-yl]-pyridine,
(Compound 50) 4-[2-(3-Chloro-phenyl)-5-(4-chloro-phenyl)-1H-imidazole-4-yl]-pyridine,
(Compound 51) 4-[5-(4-Chloro-phenyl)-2-(3-methoxy-phenyl)-1H-imidazole-4-yl]-pyridine,
(Compound 52) 4-[5-(4-Chloro-phenyl)-2-(2-methoxy-phenyl)-1H-imidazole-4-yl]-pyridine,
(Compound 53) 4-[5-(4-Chloro-phenyl)-4-pyridine-4-yl-1H-imidazole-2-yl]-benzene-1,3-diol,
(Compound 54) 4-[5-(4-Chloro-phenyl)-4-pyridine-4-yl-1H-imidazole-2-yl]-2-methoxy-phenol,
(Compound 55) 4-[2-(3-Bromo-phenyl)-5-(3-trifluoromethyl-phenyl)-1H-imidazole-4-yl]-pyridine,
(Compound 56) 4-[2-(4-Trifluoromethyl-phenyl)-5-(3-trifluoromethyl-phenyl)-1H-imidazole-4-yl]-pyridine,
(Compound 57) 4-[2-(4-Bromo-phenyl)-5-(3-trifluoromethyl-phenyl)-1H-imidazole-4-yl]-pyridine,
(Compound 58) 4-[5-(3-Iodo-phenyl)-2-(4-trifluoromethyl-phenyl)-1H-imidazole-4-yl]-pyridine,
(Compound 59) 4-[5-(4-Chloro-phenyl)-2-(4-isopropyl-phenyl)-1H-imidazole-4-yl]-pyridine,
(Compound 60) 4-[5-(4-Chloro-phenyl)-4-pyridine-4-yl-1H-imidazole-2-yl]-2,6-dimethyl-phenol,
(Compound 61) 4-[5-(4-Chloro-phenyl)-2-(2,4-Dichloro phenyl)-1H-imidazole-4-yl]-pyridine,
(Compound 62) 4-[5-(4-Chloro-phenyl)-4-pyridine-4-yl-1H-imidazole-2-yl]-benzonitrile,
(Compound 63) 4-[5-(4-Chloro-phenyl)-4-pyridine-4-yl-1H-imidazole-2-yl]-phenol,
(Compound 64) 2,6-Di-tert-butyl-4-[5-(4-chloro-phenyl)-4-pyridine-4-yl-1H-imidazole-2-yl]-phenol
(Compound 65) 4-[5-(4-Chloro-phenyl)-2-(3,4-dimethoxy-phenyl)-1H-imidazole-4-yl]-pyridine,
(Compound 66) 4-[5-(4-Chloro-phenyl)-2-(3-nitro-phenyl)-1H-imidazole-4-yl]-pyridine,
(Compound 67) 4-[5-(4-Chloro-phenyl)-2-(3,4-Dichloro phenyl)-1H-imidazole-4-yl]-pyridine,
(Compound 68) 4-[5-(4-Chloro-phenyl)-2-(4-methoxy-phenyl)-1H-imidazole-4-yl]-pyridine,
(Compound 69) 4-[5-(4-Chloro-phenyl)-4-pyridine-4-yl-1H-imidazole-2-yl]-2,6-diisopropyl-phenol,
(Compound 70) N-{4-[5-(4-Chloro-phenyl)-4-pyridine-4-yl-1H-imidazole-2-yl]-acetamide,
(Compound 71) 4-[2-(3,4-Dichloro-phenyl)-5-(3-trifluoromethyl-phenyl)-1H-imidazole-4-yl]-pyridine,
(Compound 72) 4-[2-(4-Chloro-phenyl)-5-(3-trifluoromethyl-phenyl)-1H-imidazole-4-yl]-pyridine,
(Compound 73) 4-[4-Pyridine-4-yl-5-(3-trifluoromethyl-phenyl)-1H-imidazole-2-yl]-phenol,
(Compound 74) 4-[4-Pyridine-4-yl-5-(3-trifluoromethyl-phenyl)-1H-imidazole-2-yl]-2-methoxy-phenol,
(Compound 75) 4-[2-(3-Chloro-phenyl)-5-(3-trifluoromethyl-phenyl)-1H-imidazole-4-yl]-pyridine,
(Compound 76) 4-[2-(4-Methylsulfanyl-phenyl)-5-(3-trifluoromethyl-phenyl)-1H-imidazole-4-yl]-pyridine,
(Compound 77) 3-[4-Pyridine-4-yl-5-(3-trifluoromethyl-phenyl)-1H-imidazole-2-yl]-phenol,
(Compound 78) 4-[2-(3-Bromo-phenyl)-5-(3-iodo-phenyl)-1H-imidazole-4-yl]-pyridine,
(Compound 79) 4-[5-(3-Iodo-phenyl)-4-pyridin-4-yl-1H-imidazole-2-yl]-2,6-dimethyl-phenol,
(Compound 80) 4-[2-(4-Bromo-phenyl)-5-(3-iodo-phenyl)-1H-imidazole-4-yl]-pyridine,
(Compound 81) 4-[2-(3-Chloro-phenyl)-5-(3-iodo-phenyl)-1H-imidazole-4-yl]-pyridine,
(Compound 82) 4-[2-(4-Fluoro-phenyl)-5-(3-iodo-phenyl)-H-imidazole-4-yl]-pyridine,
(Compound 83) 4-[2-Naphtalene-1-yl-5-phenyl-1H-imidazole-4-yl]-pyridine,
(Compound 84) 4-(5-Phenyl-2-styryl-1H-imidazole-4-yl]-pyridine,
(Compound 85) 4-[5-Phenyl-2-(4-trifluoromethyl-phenyl)-1H-imidazole-4-yl]-pyridine,
(Compound 86) 2-Nitro-4-(5-phenyl-4-pyridine-4-yl-1H-imidazole-2-yl)phenol,
(Compound 87) 4-[2-(3-Bromo-phenyl)-5-phenyl-1H-imidazole-4-yl]-pyridine,
(Compound 88) 2,6-Dimethyl-4-(5-phenyl-4-pyridine-4-yl-1H-imidazole-2-yl)phenol,
(Compound 89) 4-[2-(3,4-Bis-benzyloxy-phenyl)-5-phenyl-1H-imidazole-4-yl]-pyridine,
(Compound 90) 4-[2-(3,4-Dimethoxy-phenyl)-5-phenyl-1H-imidazole-4-yl]-pyridine,
(Compound 91) 4-[2-(3-Nitro-phenyl)-5-phenyl-1H-imidazole-4-yl]-pyridine,
(Compound 92) 4-[2-(4-Chloro-phenyl)-5-phenyl-1H-imidazole-4-yl]-pyridine,
(Compound 93) 2-(5-Phenyl-4-pyridine-4-yl-1H-imidazole-2-yl)-benzene-1,4-diol,
(Compound 94) 4-(5-Phenyl-4-pyridine-4-yl-1H-imidazole-2-yl)-phenol,
(Compound 95) 3-(5-phenyl-4-pyridine-4-yl-1H-imidazole-2-yl)-phenol,
(Compound 96) 4-[2-(4-Bromo-phenyl)-5-phenyl-1H-imidazole-4-yl]-pyridine,
(Compound 97) 2-Methoxy-4-(5-phenyl-4-pyridine-4-yl-1H-imidazole-2-yl)phenol,
(Compound 98) 4-[2-(4-Isopropyl-phenyl)-5-phenyl-1H-imidazole-4-yl]-pyridine,
(Compound 99) 4-[2-(2,3-Dichloro-phenyl)-5-phenyl-1H-imidazole-4-yl]-pyridine,
(Compound 100) 4-[2-(2,4-Dichloro-phenyl)-5-phenyl-1H-imidazole-4-yl]-pyridine,
(Compound 101) 4-[2-(4-Methylsulfanyl-phenyl)-5-phenyl-1H-imidazole-4-yl]-pyridine,
(Compound 102) 4-[2-(2-Chloro-phenyl)-5-phenyl-1H-imidazole-4-yl]-pyridine,
(Compound 103) 4-[2-(4-Methoxy-phenyl)-5-phenyl-1H-imidazole-4-yl]-pyridine,
(Compound 104) 4-[2-(3-Methoxy-phenyl)-5-phenyl-1H-imidazole-4-yl]-pyridine,
(Compound 105) 4-[2-(2-Methoxy-phenyl)-5-phenyl-1H-imidazole-4-yl]-pyridine,
(Compound 106) 4-[2-(3-Chloro-phenyl)-5-phenyl-1H-imidazole-4-yl]-pyridine,
(Compound 107) 2,6-Di-tert-butyl-4-(5-phenyl-4-pyridine-4-yl-1H-imidazole-2-yl)-phenol,
(Compound 108) 4-(5-Phenyl-4-pyridine-4-yl-1H-imidazole-2-yl)-benzonitrile,
(Compound 109) N-[4-(5Pphenyl-4-pyridine-4-yl-1H-imidazole-2-yl)-phenyl]-acetamide,
(Compound 110) 4-{2-[2-(2-Methoxy-phenyl)-vinyl]-5-phenyl-1H-imidazole-4-yl}-pyridine,
(Compound 111) 4-[5-(3-Iodo-phenyl)-4-pyridine-4-yl-1H-imidazole-2-yl]-phenol,
(Compound 112) 4-[2-(2,3-Dichloro-phenyl)-5-(3-iodo-phenyl)-1H-imidazole-4-yl]-pyridine,
(Compound 113) 4-[5-(4-Chloro-phenyl)-2-(4-methylsulfanyl-phenyl)-1H-imidazole-4-yl]-pyridine,
(Compound 114) 4-[5-(4-Chloro-phenyl)-4-pyridine-4-yl-1H-imidazole-2-yl]-dimethyl amine,
(Compound 115) 4-[5-(3-Iodo-phenyl)-2-(5-methyl-furan-2-yl)-1H-imidazole-4-yl]-pyridine,
(Compound 116) 4-[4-(4-Fluoro-phenyl)-5-pyridine-4-yl-1H-imidazole-2-yl]-benzylamine,
(Compound 117) 4-[5-(3-Iodo-phenyl)-2-(4-methylsulfanylphenyl)-3H-imidazole-4-yl]-pyridine,
(Compound 118) 4-[2-(4-Methanesulfinyl-phenyl)-5-phenyl-3H-imidazole-4-yl]-pyridine,
(Compound 119) 4-[5-(4-Fluoro-phenyl)-4-pyridin-4-yl-1H-imidazole-2-yl]-phenylamine,
(Compound 120) {4-[5-(3-Iodo-phenyl)-4-pyridin-4-yl-1H-imidazole-2-yl]-phenyl}-methanol,
(Compound 121) 4-[5-(4-Fluoro-phenyl)-4-pyridin-4-yl-1H-imidazole-2-yl]-benzylamine,
(Compound 122) 2-(3,4-Dimethoxyphenyl)-4,5-bis-(4-methoxyphenyl)-1H-imidazole,
(Compound 123) 4-[4,5-Bis-(4-methoxyphenyl)-1H-imidazole-2-yl]-2,6-bis-tert-butyl-phenol,
(Compound 124) 4-[4,5-Bis-(4-methoxyphenyl)-1H-imidazole-2-yl]-2-methoxy-phenol,
(Compound 125) 4-[4,5-Bis-(4-bromophenyl)-1H-imidazole-2-yl]-2-methoxy-phenol,
(Compound 126) 4-[4,5-Bis-(4-methoxyphenyl)-2-styryl-1H-imidazole,
(Compound 127) 4-[4,5-Bis-(4-methoxyphenyl)-2-(4-trifluoromethyl-phenyl)-1H-imidazole],
(Compound 128) 4-[4,5-Bis-(4-methoxyphenyl)-2-(3-trifluoromethyl-phenyl)-1H-imidazole,
(Compound 129) 4-[4,5-Bis-(4-methoxyphenyl)-1H-imidazole-2-yl]-2-nitro-phenol,
(Compound 130) 4-[4,5-Bis-(4-methoxyphenyl)-1H-imidazole-2-yl]-4-nitro-phenol,
(Compound 131) 4-[4,5-Bis-(4-methoxyphenyl)-1H-imidazole-2-yl]-phenol,
(Compound 132) 2-(3-Bromo-phenyl)-4,5-bis-(4-methoxyphenyl)-1H-imidazole,
(Compound 132) 2-(3,4-Diphenoxy-phenyl)-4,5-bis-(4-methoxyphenyl)-1H-imidazole,
(Compound 133) {4-[4,5-Bis-(4-methoxyphenyl)-1H-imidazole-2-yl]-phenyl}-dimethylamine,
(Compound 134) 2-(4-Chloro-phenyl)-4,5-bis-(4-methoxyphenyl)-1H-imidazole,
(Compound 135) 2-(4-Bromo-phenyl)-4,5-bis-(4-methoxyphenyl)-1H-imidazole,
(Compound 136) 4,5-Bis-(4-methoxyphenyl)-2-(3-nitrophenyl)-1H-imidazole,
(Compound 137) 4,5-Bis-(4-methoxyphenyl)-2-naphthalen-1-yl-1H-imidazole,
(Compound 138) 2-(2,3-Dichlorophenyl)-4,5-bis-(4-methoxyphenyl)-1H-imidazole,
(Compound 139) 2-(2,4-Dichlorophenyl)-4,5-bis-(4-methoxyphenyl)-1H-imidazole,
(Compound 140) 4,5-Bis-(4-methoxyphenyl)-2-(4-nitro-phenyl)-1H-imidazole,
(Compound 141) 4-[4,5-Bis-(4-methoxyphenyl)-1H-imidazole-2-yl]-benzene-1,2-diol,
(Compound 142) 2-(4-Methoxy-3,5-dimethyl-phenyl)-4,5-bis-(4-methoxyphenyl)-1H-imidazole,
(Compound 143) 4-[4,5-Bis-(4-methoxyphenyl)-1H-imidazole-2-yl]-1H-indole,
(Compound 144) 2-(3,4-Bis-benzyloxy-phenyl)-4,5-bis-(4-bromo-phenyl)-1H-imidazole,
(Compound 145) 4,5-Bis-(4-bromo-phenyl)-2-(4-isopropyl-phenyl)-1H-imidazole,
(Compound 146) 4,5-Bis-(4-bromo-phenyl)-2-(2,4-dichloro-phenyl)-1H-imidazole,
(Compound 147) 4,5-Bis-(4-bromo-phenyl)-2-(4-chloro-phenyl)-1H-imidazole,
(Compound 148) 4,5-Bis-(4-bromo-phenyl)-2-(4-trifluoromethyl-phenyl)-1H-imidazole,
(Compound 149) 4,5-Bis-(4-bromo-phenyl)-2-(3-trifluoromethyl-phenyl)-1H-imidazole,
(Compound 150) 2-(3,5-Bis-trifluoromethyl-phenyl)-4,5-bis-(4-bromo-phenyl)-1H-imidazole,
(Compound 151) 2-(3,5-Bis-trifluoromethyl-phenyl)-4,5-bis-(4-bromo-phenyl)-1H-imidazole,
(Compound 152) 4,5-Bis-(4-bromo-phenyl)-2-(3,4-dimethoxy-phenyl)-1H-imidazole,
(Compound 153) 4,5-Bis-(4-bromo-phenyl)-2-(4-methylsulfanyl-phenyl)-1H-imidazole,
(Compound 154) 2-(3-Bromo-phenyl)-4,5-bis-(4-bromo-phenyl)-1H-imidazole,
(Compound 155) 4,5-Bis-(4-bromo-phenyl)-2-(2,3-dichloro-phenyl)-1H-imidazole,
(Compound 156) 4,5-Bis-(4-bromo-phenyl)-2-(3-nitro-phenyl)-1H-imidazole,
(Compound 157) 4-[4,5-Bis-(4-bromo-phenyl)-1H-imidazole-2-yl]-2,6-dimethyl-phenol,
(Compound 158) 4,5-Bis-(4-bromo-phenyl)-2-(4,5-dimethoxy-2-nitro-phenyl)-1H-imidazole,
(Compound 159) 4-[4,5-Bis-(4-bromo-phenyl)-1H-imidazole-2-yl]-2-nitro-phenol,
(Compound 160) {4-[4,5-Bis-(4-bromo-phenyl)-1H-imidazole-2-yl]-phenyl}-dimethylamine,
(Compound 161) 4,5-Bis-(4-bromo-phenyl)-2-naphthalen-1-yl-1H-imidazole,
(Compound 162) 4,5-Bis-(4-bromo-phenyl)-2-(5-ethyl-furan-2-yl)-1H-imidazole,
(Compound 163) 4,5-Bis-(4-bromo-phenyl)-2-thiophen-2-yl-1H-imidazole,
(Compound 164) 3-[4,5-Bis-(4-bromophenyl)-1H-imidazole-2-yl]-1H-indole,
(Compound 165) 2-(3,4-Dimethoxy-phenyl)-4,5-di-thiophen-2-yl-1H-imidazole,
(Compound 166) 2-(4-Isopropyl-phenyl)-4,5-di-thiophen-2-yl-1H-imidazole,
(Compound 167) 2-(3-Bromo-phenyl)-4,5-di-thiophen-2-yl-1H-imidazole,
(Compound 168) 4,5-Di-thiophen-2-yl-2-(4-trifluoromethyl-phenyl)-1H-imidazole,
(Compound 169) 4,5-Di-thiophen-2-yl-2-(3-trifluoromethyl-phenyl)-1H-imidazole,
(Compound 170) [4-(4,5-Di-thiophen-2-yl-1H-imidazole-2-yl)-phenyl]-dimethylamine,
(Compound 171) 2-(3,4-Bis-benzyloxy-phenyl)-4,5-di-thiophen-2-yl-1H-imidazole,
(Compound 172) 2-Naphthalen-1-yl-4,5-di-thiophen-2-yl-1H-imidazole,
(Compound 173) 4-(4,5-Di-thiophen-2-yl-1H-imidazole-2-yl)-2-nitrophenol.

11. The compound according to claim 1, which compound is 4-[4-(4-fluorophenyl)-5-pyridine-4-yl-1H-imidazole-2-yl]-phenol, 4-[5-(3-iodo-phenyl)-2-(4-methanesulfinyl-phenyl)-3H-imidazole-4-yl]-pyridine, or 4-[4-(4-fluoro-phenyl)-5-pyridine-4-yl-1H-imidazole-2-yl]-benzylamine.

12. A method for identifying compounds useful for the prophylaxis and/or treatment of Hepatitis C Virus (HCV) infections and/or diseases comprising the steps:
a) contacting a human cellular protein selected from the group consisting of casein kinase 1 alpha (a), delta (a), and epsilon (6) with a compound to be tested; and
b) determining the activity of the human cellular protein.

13. The method according to claim 12, wherein the compound to be tested is a monoclonal or polyclonal antibody that has the capability of binding to the human cellular protein.

14. The method according to claim 12, wherein the compound to be tested is an imidazole compound falling under the compounds recited in claims 1.

15. The method according to claim 14, wherein the imidazole compound is 4-[4-(4-fluoro-phenyl)-5-pyridine-4-yl-1H-imidazole-2-yl]-phenol, 4-[5-(3-iodo-phenyl)-2-(4-methanesulfinyl-phenyl)-3H-imidazole-4-yl]-pyridine, or 4-[4-(4-fluoro-phenyl)-5-pyridine-4-yl-1H-imidazole-2-yl]-benzylamine.

16. A method for detecting a Hepatitis C Virus infection and/or a disease associated therewith in an individual, the method comprising the following steps:
a) providing a sample of the individual; and
b) determining the activity, in the sample, of one or more proteins selected from the group consisting of casein kinase 1 alpha (α), delta (δ), and epsilon (ε).

17. A method for detecting a Hepatitis C Virus infection and/or a disease associated therewith in cells and/or a cell lysate, the method comprising the following steps:
a) providing a sample of the cells or the cell lysate; and
b) determining the activity, in the sample, of one or more proteins selected from the group consisting of casein kinase 1 alpha (a), delta (6), and epsilon (E).

18. A monoclonal or polyclonal antibody that has the capability to bind to a human cellular protein selected from the group consisting of casein kinase I α, δ, and ε.

19. A method for preventing and/or treating HCV infections and/or diseases in an individual comprising the step of administering to the individual a pharmaceutically effective amount of an agent which inhibits at least partially the activity and/or production of at least one of the human cellular proteins selected from the group consisting of casein kinase I α, δ, and ε.

20. A method for regulating the production and/or replication of HCV in an individual comprising the step of administering to the individual a pharmaceutically effective amount of an agent which inhibits at least partially the activity and/or production of at least one of the human cellular proteins selected from the group consisting of casein kinase I α, δ, and ε.

21. (canceled)

22. The method according to one of claim 19, wherein the agent is an imidazole compound falling under the general formula (I) or a monoclonal or polyclonal antibody that has the capability to bind to a human cellular protein selected from the group consisting of casein kinase I α, δ, and ε.

23. The method according to claim 20, wherein the agent is an imidazole compound falling under the general formula (I) or a monoclonal or polyclonal antibody that has the capability to bind a human cellular protein selected from the group consisting of casein kinase I α, δ, and ε.

24. The method according to claim 23, wherein the imidazole compound is 4-[4-(4-fluoro-phenyl)-5-pyridine-4-yl-1H-imidazole-2-yl]-phenol, 4-[5-(3-iodo-phenyl)-2-(4-methanesulfinyl-phenyl)-3H-imidazole-4-yl]-pyridine, or 4-[4-(4-fluoro-phenyl)-5-pyridine-4-yl-1H-imidazole-2-yl]-benzylamine.

25. An oligonucleotide that has the capability to bind to DNA and/or RNA encoding a human cellular protein selected from the group consisting of casein kinase I α, δ, and ε.

26. A method for regulating the expression of a human cellular protein selected from the group consisting of casein kinase I α, δ, and ε in an individual, comprising the step of administering to the individual a pharmaceutically effective amount of an agent which inhibits at least partially the transcription of DNA or the translation RNA encoding the human cellular protein selected from the group consisting of casein kinase I α, δ, and ε.

27. A method for regulating the expression of a human cellular protein selected from the group consisting of casein kinase I α, δ, and ε in a cell, comprising the step of administering to the cell a pharmaceutically effective amount of an agent which inhibits at least partially the transcription of DNA or the translation RNA encoding the human cellular protein selected from the group consisting of casein kinase I α, δ, and ε.

28. The method according to claim 19, wherein the agent is an oligonucleotide which has the capability to bind to DNA and/or RNA encoding fully or partially at least one of the human cellular proteins selected from the group consisting of casein kinase I α, δ, and ε.

29. A solid support useful for screening compounds which are capable of prophylaxis and/or treatment of HCV infections and/or diseases in an individual, the solid support comprising at least one immobilized oligonucleotide encoding the human cellular proteins selected from the group consisting of casein kinase I α, δ, and ε.

30. A solid support useful for screening compounds which are capable of prophylaxis and/or treatment of HCV infections and/or diseases in an individual, the solid support comprising at least one immobilized human cellular protein selected from the group consisting of casein kinase I α, δ, and ε.

31. A pharmaceutical composition comprising at least one agent capable of inhibiting at least partially the activity of at least one human cellular protein selected from the group consisting of casein kinase I α, δ, and ε.

32. The pharmaceutical composition according to claim 31, wherein the agent is a monoclonal and/or polyclonal antibody.

33. The pharmaceutical composition according to claim 31, wherein the agent is an oligonucleotide which can bind to the DNA and/or RNA encoding at least one of the human cellular proteins selected from the group consisting of casein kinase I α, δ, and ε.

34. The pharmaceutical composition according to claim 31, wherein the agent is an imidazole compound falling under general formula (I).

35. The pharmaceutical composition according to claim 34, wherein the imidazole compound is 4-[4-(4-fluoro-phenyl)-5-pyridine-4-yl-1H-imidazole-2-yl]-phenol, 4-[5-(3-iodo-phenyl)-2-(4-methanesulfinyl-phenyl)-3H-imidazole-4-yl]-pyridine, or 4-[4-(4-fluoro-phenyl)-5-pyridine-4-yl-1H-imidazole-2-yl]-benzylamine.

36. The pharmaceutical composition according to claim 34, further comprising alpha interferon.

37. The pharmaceutical composition according to claim 34, further comprising ribavirin.

38. The pharmaceutical composition according to claim 34, further comprising pegylated interferon.

39-46. (canceled)

47. The method according to claim 22, wherein the imidazole compound is 4-[4-(4-fluoro-phenyl)-5-pyridine-4-yl-1H-imidazole-2-yl]-phenol, 4-[5-(3-iodo-phenyl)-2-(4-methanesulfinyl-phenyl)-3H-imidazole-4-yl]-pyridine, or 4-[4-(4-fluoro-phenyl)-5-pyridine-4-yl-1H-imidazole-2-yl]-benzylamine.

48. The method according to claim 20, wherein the agent is an oligonucleotide which has the capability to bind to DNA and/or RNA encoding fully or partially at least one of the human cellular proteins selected from the group consisting of casein kinase I α, δ, and ε.

49. The method according to claim 26, wherein the agent is an oligonucleotide which has the capability to bind to DNA and/or RNA encoding fully or partially at least one of the human cellular proteins selected from the group consisting of casein kinase I α, δ, and ε.

50. The method according to claim 27, wherein the agent is an oligonucleotide which has the capability to bind to DNA and/or RNA encoding fully or partially at least one of the human cellular proteins selected from the group consisting of casein kinase I α, δ, and ε.
